(19) United States
(12) Patent Application Publication
(10) Pub. No.: US 2019/0338269 A1
(43) Pub. Date: Nov. 7, 2019

(54) FACTOR X VARIANTS

(71) Applicant: LABORATOIRE FRANCAIS DU FRACTIONNEMENT ET DES BIOTECHNOLOGIES, LES ULIS (FR)

(72) Inventors: Abdessatar Sami CHTOUROU, ELANCOURT (FR); Jean-Luc PLANTIER, CROIX (FR); Toufik ABACHE, SANTES (FR)

(21) Appl. No.: 16/099,297

(22) PCT Filed: May 5, 2017
(86) PCT No.: PCT/FR2017/051094

§ 371 Date: Nov. 6, 2018

(30) Foreign Application Priority Data
May 6, 2016 (FR) 1654098

(51) Int. Cl. C12N 9/64; A61K 38/48; C12N 15/62; C12N 15/85

(52) U.S. Cl. CPC C12N 9/6432; A61K 38/48; C12N 2015/8518; C12N 15/85; C12N 15/62

(57) ABSTRACT

Disclosed is a protein that is a factor X variant including a mutated sequence of SEQ ID NO:1; at its N-terminus, the protein includes the signal peptide of sequence SEQ ID NO:7 and a propeptide that differs from the natural factor X propeptide.

The present invention relates to factor X variants, and to the use thereof for treating blood coagulation disorders.

Factor X is a protein present in the blood. This protein plays an important role in the coagulation cascade. Blood coagulation is a complex process which makes it possible to prevent blood flow via damaged vessels. As soon as a vessel is broken, the elements responsible for coagulation interact with one another to form a plug, the platelet plug, at the site where the vessel is broken. The coagulation factors are required in order to hold the platelet plug in place and to stabilize the clot.

The formation of a normal clot occurs in four steps:

Step 1 The blood vessel is damaged.

Step 2 The blood vessel contracts so as to restrict the blood supply to the damaged zone.

Step 3 The platelets adhere to the subendothelial space exposed during the damaging of the vessel and also to the stimulated blood vessel walls. The platelets spread, and this is referred to as “platelet adhesion”. These spread platelets release substances which activate other neighboring platelets such that they agglomerate at the site of the lesion in order to form the platelet plug. This is known as “platelet aggregation”.

Step 4 The surface of the activated platelets thus constitutes a surface on which blood coagulation can take place. The coagulation proteins which circulate in the blood (including factor X) are activated at the surface of the platelets and form a fibrin clot.

These coagulation proteins (i.e. factors I, II, V, VIII, IX, X, XI, XII and XIII, and also von Willebrand factor) operate in a chain reaction, i.e. the coagulation cascade.

Factor X in activated form (Xa) is involved more particularly in the activation of prothrombin (factor II) to thrombin (factor IIa), in particular when it is complexed with activated cofactor V so as to form the prothrombinase complex. This factor is an essential element in the coagulation cascade. When this factor is lacking, bleeding occurs, such as epistaxis (nose bleeds), hemarthrosis (effusion of blood into a joint cavity) or gastrointestinal bleeding. Factor X deficiency is extremely rare. Its transmission is autosomic recessive, and its prevalence is 1/1 000 000.

Fx activation occurs:
either very early during the step of initiation of the coagulation cascade by the factor VIIa/tissue factor complex, in a relatively ineffective reaction which results in the formation of traces of thrombin;
or during the step of amplification of the coagulation cascade resulting from positive feedback produced by the traces of thrombin, resulting in the activation of factors VIII and IX.

The latter two factors are missing in individuals suffering from hemophilia A and B, thus causing a hemorrhagic disorder which can be fatal without treatment. The absence of these factors means that it is not possible to generate sufficient amounts of activated factor X to stop the hemorrhage.

The first 42 amino acids of the light chain of factor X (residues 1-42 of SEQ ID No.: 5) represent the “Gla” domain, which is the phospholipid-binding domain. The “Gla” domain contains 11 glutamic acid (Glu) residues all or some of which being post-translationally modified (gamma-carboxylated) to give γ-carboxyglutamic acids (Gla). Factor X is thus a coagulation protein of which the biological activity depends on the degree of gamma-carboxylation of its “Gla” domain.

All of the “Gla” proteins or Gla-domain proteins are dependent on vitamin K. Vitamin K is a liposoluble vitamin involved in the gamma-carboxylation of glutamate protein residues so as to form gamma-carboxyglutamate residues. The gamma-carboxyglutamate residues are essential for the biological activity of all proteins which have Gla domains, in particular via a high calcium ion-binding affinity.

The presence of gamma-carboxylated Glu residues (also called Gla residues) in vitamin K-dependent proteins has thus proved to be essential for their functional activation. Thus, the presence of Glu residues on factor X and their level of gamma-carboxylation is essential to the functional activity of activated factor X.

There is thus a need for a modified factor X which can be activated by thrombin, and which has a degree of gamma-carboxylation that would make it possible to have efficient coagulation in the absence of factor VIII and/or of factor IX, through the direct use of the traces of thrombin generated during the initiation of coagulation.

The inventors have identified specific factor X mutants (also called factor X variants), which are efficiently activated by thrombin, thus making it possible to restore coagulation in the absence of factor VIII and of factor IX. Preferably, the specific factor X mutants identified by the inventors can also restore coagulation in the absence of factor X. Indeed, as demonstrated in the examples, these factor X mutants can be activated by thrombin, and allow efficient coagulation, even in the absence of endogenous factor VIII and/or factor IX and/or factor X.

These factor X mutants advantageously exhibit a high degree of gamma-carboxylation. The term “high degree of gamma-carboxylation” is intended to mean a degree of gamma-carboxylation at least equal to 20%, preferably at least equal to 25%, 30%, 35%, 40%, 45%, 50%, 55%, 60%, 65%, 70%, 75%, 80%, 85%, 90%, 95% of the gamma-carboxylation of plasma factor X, considered to be 100%.

The present invention relates to a protein which is a factor X variant comprising a mutated sequence of SEQ ID No.: 1, said protein comprising, at its N-terminal end, the natural signal peptide of factor X, represented by the sequence SEQ ID No.: 7, and a propeptide different than the natural propeptide of factor X.

The present invention relates to a protein comprising a mutated sequence of SEQ ID No.: 1, said mutated sequence of SEQ ID No.: 1 comprising a mutation A, A′, B, C or C′, wherein:

the mutation A consists of the substitution of amino acids 43 to 52 of the sequence SEQ ID No.: 1 by a sequence chosen from DFLAEGLTPR, KATN*ATLSPR and KATXATLSPR,

the mutation A′ consists of the substitution of amino acids 47 to 52 of the sequence SEQ ID No.: 1 by a sequence chosen from TSKLTR, FNDFTR, LSSMTR, PPSLTR and LSCGQR,

the mutation B consists of the insertion of a sequence chosen from DFLAEGLTPR, KATN*ATLSPR, KATXATLSPR, TSKLTR, FNDFTR, LSSMTR, PPSLTR and LSCGQR, between amino acids 52 and 53 of the sequence SEQ ID No.: 1,

the mutation C consists of the insertion of a sequence chosen from DFLAEGLTPR, KATN*ATLSPR and KATXATLSPR, between amino acids 52 and 53 of the sequence SEQ ID No.: 1, and of the deletion of amino acids 4 to 13 of the sequence SEQ ID No.: 1,

the mutation C′ consists of the insertion of a sequence chosen from TSKLTR, FNDFTR, LSSMTR, PPSLTR and LSCGQR, between amino acids 52 and 53 of the sequence SEQ ID No.: 1, and of the deletion of amino acids 4 to 9 of the sequence SEQ ID No.: 1,

wherein N* is an optionally glycosylated asparagine, and

said protein comprising, at its N-terminal end, the signal peptide of sequence SEQ ID No.: 7 fused to a propeptide different than the natural propeptide of factor X.

Preferably, the present invention relates to a protein comprising a mutated sequence of SEQ ID No.: 1, said mutated sequence of SEQ ID No.: 1 comprising a mutation consisting of the insertion of a sequence chosen from DFLAEGLTPR, KATN*ATLSPR, KATXATLSPR, TSKLTR, FNDFTR, LSSMTR, PPSLTR and LSCGQR, between amino acids 52 and 53 of the sequence SEQ ID No.: 1 (i.e. the mutation B above),

wherein N* is an optionally glycosylated asparagine, and

said protein comprising, at its N-terminal end, the signal peptide of sequence SEQ ID No.: 7 fused to a propeptide of a coagulation factor different than factor X.

Such a protein according to the invention is a mutated factor X, which is effective in the treatment of coagulation disorders.

In particular, a sequence encoding a mutated factor X according to the invention and comprising a combination of sequences from the N-terminal to C-terminal end, namely:
a propeptide different than the natural propeptide of factor X,
the signal peptide of sequence SEQ ID No.: 7,
a mutated sequence of SEQ ID No.: 1 according to the invention,

makes it possible to directly have an impact on the gamma-carboxylation of the factor X expressed, which gamma-carboxylation is increased compared with a factor X comprising a mutated sequence of SEQ ID No.: 1 according to the invention, but not comprising, at its N-terminal end, the signal peptide of sequence SEQ ID No.: 7 fused to a propeptide of a coagulation factor different than factor X.

Preferably, such factor X mutants exhibit a degree of gamma-carboxylation at least equal to 50%, 55%, 60%, 65%, 70%, 75%, 80%, 85%, 90%, 95% of the gamma-carboxylation of plasma factor X, considered to 100%.

Another subject of the invention is a polynucleotide encoding said protein.

Another subject of the invention is an expression vector comprising said polynucleotide.

Another subject of the invention is a host cell comprising said expression vector or said polynucleotide.

Another subject of the invention is the use of said protein as a medicament. In particular, said protein can be used for the treatment of blood coagulation disorders, in particular hemorrhagic disorders, such as hemophilias A, B and C (factor XI deficiency), or factor X deficiencies, or even emergency coagulation needs in order to substitute for factor VIIa. When a powerful and rapid procoagulant response is required, said protein can be used in combination with other hemostatic molecules, such as factor VIIa and/or fibrinogen, or even in combination with procoagulant compounds (platelet transfusion, procoagulant mixtures such as FEIBA, Kaskadil, Kanokad, etc.), which will be able to reinforce the efficacy of the treatment.

The term “protein” refers to an amino acid sequence having more than 100 amino acids. Preferably, the protein consists of an amino acid sequence having between 100 and 1000 amino acids, preferably between 120 and 600 amino acids.

A factor X variant according to the invention advantageously exhibits a high degree of gamma-carboxylation. The term “high degree of gamma-carboxylation” is intended to mean a degree of gamma-carboxylation at least equal to 20%, preferably at least equal to 25%, 30%, 35%, 40%, 45%, 50%, 55%, 60%, 65%, 70%, 75%, 80%, 85%, 90%, 95% of the gamma-carboxylation of plasma factor X, considered to be 100%.

Said degree of gamma-carboxylation can be calculated by any conventional technique which makes it possible to detect and then quantify Gla residues, such as the ELISA technique using an anti-Gla antibody for capturing the gamma-carboxylated factors X. For example, the optical density measurement obtained for a variant factor X according to the invention can be related to the optical density measurement obtained for one and the same amount of plasma factor X obtained according to the same production method, considered to be the reference measurement of the degree of gamma-carboxylation at 100%.

Preferably, a factor X variant according to the invention comprises at least 2 gamma-carboxylated Glu residues (i.e. Gla residues) among 11 Glu, at least 3 gamma-carboxylated residues among 11 Glu, at least 4 gamma-carboxylated residues among 11 Glu, at least 5 gamma-carboxylated residues among 11 Glu, at least 6 gamma-carboxylated residues among 11 Glu, at least 7 gamma-carboxylated residues among 11 Glu, at least 8 gamma-carboxylated residues among 11 Glu, at least 9 gamma-carboxylated residues among 11 Glu, at least 10 gamma-carboxylated residues among 11 Glu, or 11 gamma-carboxylated residues. Preferentially, a factor X variant according to the invention comprises at least 10 of the 11 abovementioned gamma-carboxylatable residues, present on the Gla domain of the factor X light chain, which are gamma-carboxylated. Thus, preferably, a factor X variant according to the invention comprises 10 gamma-carboxylated Glu residues (10 Gla residues). More preferentially, a factor X variant according to the invention comprises 11 gamma-carboxylated Glu residues (11 Gla residues).

According to one particular aspect, the invention relates to a composition of factor X variants according to the invention, advantageously exhibiting a high degree of gamma-carboxylation within the composition. The expression “high degree of gamma-carboxylation within the composition” is intended to mean a degree of gamma-carboxylation at least equal to 20%, preferably at least equal to 25%, 30%, 35%, 40%, 45%, 50%, 55%, 60%, 65%, 70%, 75%, 80%, 85%, 90%, 95% of the gamma-carboxylation within a composition of plasma factor X, considered to be 100%.

In this case, a composition of factor X variants according to the invention can comprise a population of gamma-carboxylated factors X which is homogeneous with respect to the number of Gla residues. Preferably, a composition of factor X variants according to the invention comprises a population of gamma-carboxylated factors X, each comprising 10 Gla residues. Preferably, a composition of factor X variants according to the invention comprises a population of gamma-carboxylated factors X, each comprising 11 Gla residues. Alternatively, a composition of factor X variants according to the invention can comprise a heterogeneous population of gamma-carboxylated factors X, comprising at least two populations of gamma-carboxylated factors X not comprising the same number of gamma-carboxylated Gla residues. For example, a composition of factor X variants according to the invention can comprise 50% of variants comprising 10 Gla residues and 50% of variants comprising 11 Gla residues.

Factor X, also known as Stuart-Power factor, is encoded by the F10 gene and refers to the serine protease EC3.4.21.6. Factor X is composed of a heavy chain, of 306 amino acids, and of a light chain, of 139 amino acids.

Factor X is a protein of 488 amino acids, consisting of a signal peptide, a propeptide and light and heavy chains.

Human factor X can be found in UniProtKB under accession number P00742. Its primary structure is illustrated in FIG. 1.

The protein is translated in prepropeptide form. After cleavage of the signal peptide, the propeptide is finally cleaved, resulting in a light chain and a heavy chain (respectively of 142 and 306 amino acids) (zymogen). Following the triggering of coagulation, the heavy chain is finally activated by cleavage of the activation peptide, so as to contain only 254 amino acids (the first 52 amino acids are cleaved during the processing): it is the heavy chain of factor Xa (SEQ ID No.: 6).

The prepropeptide of human factor X corresponds to SEQ ID No.: 4. The heavy chain of non-activated human factor X corresponds to SEQ ID No.: 1, and the light chain corresponds to SEQ ID No.: 5. The heavy chain activation peptide corresponds to SEQ ID No.: 3, and comprises 52 amino acids. The signal peptide corresponds to SEQ ID No.: 7, and comprises 31 amino acids. The natural propeptide of factor X corresponds to SEQ ID No.: 8, and comprises 9 amino acids.

SEQ ID No.: 2 is identical to amino acids 1 to 182 of SEQ ID No.: 4.

SEQ ID No.: 1 is identical to amino acids 183 to 488 of SEQ ID No.: 4.

The heavy chain of factor Xa (SEQ ID No.: 6) corresponds to SEQ ID No.: 1, wherein the activation peptide represented by SEQ ID No.: 3 has been cleaved.

In the context of the invention, the expression “natural propeptide of factor X” is preferably intended to mean a variant of the natural propeptide of human factor X represented by the sequence SEQ ID No.: 9 which comprises 9 amino acids.

The protein according to the invention is a factor X mutant (or variant).

The preferred mutation according to the invention consists of the insertion of a sequence chosen from DFLAEGLTPR, KATN*ATLSPR, KATXATLSPR, TSKLTR, FNDFTR, LSSMTR, PPSLTR and LSCGQR, between amino acids 52 and 53 of the sequence SEQ ID No.: 1 (“mutation B”), wherein N* is an optionally glycosylated asparagine. Preferably, the mutation according to the invention consists of the insertion of the sequence DFLAEGLTPR between amino acids 52 and 53 of the sequence SEQ ID No.: 1.

The sequence SEQ ID No.: 1 comprising a mutation according to the invention is also called “mutated sequence of SEQ ID No.: 1”.

In other words, preferably, the sequence SEQ ID No.: 1 comprising a mutation according to the invention consists in the sequence SEQ ID No.: 3, fused, at its C-terminal end, to a sequence chosen from DFLAEGLTPR, KATN*ATLSPR, KATXATLSPR, TSKLTR, FNDFTR, LSSMTR, PPSLTR and LSCGQR (“mutation B”), wherein N* is an optionally glycosylated asparagine, itself fused, at its C-terminal end, to the sequence SEQ ID No.: 6.

When the mutation consists of the insertion of the sequence DFLAEGLTPR, the mutated sequence of SEQ ID No.: 1 corresponds to SEQ ID No.: 11.

The propeptide used in the protein according to the invention is different than the natural propeptide of factor X. Preferably, the propeptide used in the protein according to the invention is that of a vitamin K-dependent protein. Preferably, the propeptide used in the protein according to the invention is chosen from the protein S propeptide, the protein Z propeptide, the FIX propeptide, the propeptide of one of the proteins GAS6, BGP, MGP, PRGP1, PRGP2, TMG3 and TMG4, the thrombin propeptide, the factor VII propeptide, and the protein C propeptide, including the natural isoforms thereof or the modified versions thereof. The term “modified version” is intended to mean that the propeptide used is truncated, and optionally comprises the insertion of one or more amino acids, for example at its N-terminal end. Preferably, the propeptide used in the protein according to the invention is that of a coagulation factor different than factor X. Thus, the propeptide used in the protein according to the invention is preferentially the natural propeptide of a coagulation factor different than factor X. Preferably, the propeptide is chosen from the thrombin propeptide, the factor VII propeptide, and the protein C propeptide, and the natural isoforms thereof or modified versions thereof. Preferably, the factor VII propeptide according to the invention corresponds to the A isoform of the factor VII propeptide (or “FVIIv1”) and has the sequence SEQ ID No.: 14. Preferably, the factor VII propeptide according to the invention corresponds to the B isoform of the factor VII propeptide (or “FVIIv2”) and has the sequence SEQ ID No.: 15.

More preferentially, the propeptide is chosen from the sequences SEQ ID No.: 13, SEQ ID No.: 14, SEQ ID No.: 15 and SEQ ID No.: 16. More preferentially, the propeptide has the sequence SEQ ID No.: 14.

Preferably, the signal peptide of sequence SEQ ID No.: 7 fused to a propeptide different than the natural propeptide of factor X is chosen from the sequences SEQ ID No.: 18, SEQ ID No.: 19, SEQ ID No.: 20 and SEQ ID No.: 21. More preferentially, the signal peptide of sequence SEQ ID No.: 7 fused to a propeptide different than the natural propeptide of factor X is represented by the sequence SEQ ID No.: 19.

The particular combination of the sequence SEQ ID No.: 19 to a mutated sequence of SEQ ID No.: 1 according to the invention makes it possible to directly have an impact on the gamma-carboxylation of the mutated factor X comprising such a combination, compared with a mutated factor X comprising a mutated sequence of SEQ ID No.: 1 according to the invention, but not comprising, at its N-terminal end, the sequence SEQ ID No.: 19.

The protein according to the invention preferably comprises an intermediate sequence, between the signal peptide of sequence SEQ ID No.: 7 fused to a propeptide other than the natural propeptide of factor X, and the mutated sequence of SEQ ID No.: 1. Preferably, said intermediate sequence is fused, at its N-terminal end, to the signal peptide of sequence SEQ ID No.: 7 fused to a propeptide different than the natural propeptide of factor X, and, at its C-terminal end, to the mutated sequence of SEQ ID No.: 1. Preferably, said intermediate sequence is the sequence of the factor X light chain, preferably the sequence SEQ ID No.: 5.

The protein according to the invention thus preferably comprises, from the N-terminal to C-terminal end:
the signal peptide of sequence SEQ ID No.: 7 fused to a propeptide different than the natural propeptide of factor X, then
the sequence SEQ ID No.: 5, then
said mutated sequence of SEQ ID No.: 1.

More particularly, the protein according to the invention comprises, in the N-terminal to C-terminal direction, the signal peptide of sequence SEQ ID No.: 7 fused to a propeptide different than the natural propeptide of factor X, then the sequence SEQ ID No.: 5, then the mutated sequence of SEQ ID No.: 1.

The protein according to the invention preferably comprises, from the N-terminal to C-terminal end:
the signal peptide of sequence SEQ ID No.: 7 fused to a propeptide different than the natural propeptide of factor X, then
the sequence SEQ ID No.: 5, then
the sequence SEQ ID No.: 11.

Thus, the protein according to the invention preferably comprises, from the N-terminal to C-terminal end: the signal peptide of sequence SEQ ID No.: 7 fused to a propeptide different than the natural propeptide of factor X, fused to the sequence SEQ ID No.: 17.

Preferably, the protein according to the invention comprises, preferably consists of, a sequence chosen from SEQ ID No.: 22, SEQ ID No.: 23, SEQ ID No.: 24 and SEQ ID No.: 25. More preferentially, the protein according to the invention comprises, preferably consists of, the sequence SEQ ID No.: 23.

The mutated sequence of SEQ ID No.: 1 comprising a mutation A, A′, B, C or C′ can also comprise at least one mutation of at least one amino acid which does not impair the functional activity of the protein according to the invention. Preferably, the mutated sequence of SEQ ID No.: 1 comprising a mutation A, A′, B, C or C′ and also comprising at least one additional mutation of at least one amino acid which does not impair the functional activity, exhibits at least 80% identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity with the mutated sequence of SEQ ID No.: 1 comprising only a mutation A, A′, B, C or C′.

The protein according to the invention can also be fused, in the C-terminal position, to at least one wild-type immunoglobulin fragment, which is optionally mutated. The term “wild-type immunoglobulin fragment” is intended to mean a fragment chosen from the wild-type Fc fragments and the wild-type scFc fragments, which are optionally mutated.

The term “Fc fragment” is intended to mean the constant region of a full-length immunoglobulin with the exclusion of the first immunoglobulin constant region domain (i.e. CH1-CL). Thus, the Fc fragment refers to a homodimer, each monomer comprising the last two constant domains of IgA, IgD, IgG (i.e. CH2 and CH3), or the last three constant domains of IgE and IgM (i.e. CH2, CH3 and CH4), and the N-terminal flexible hinge region of these domains. The Fc fragment, when it is derived from IgA or from IgM, can comprise the J chain. Preferably, the Fc region of an IgG1 is composed of the N-terminal flexible hinge and the CH2-CH3 domains, i.e. the portion starting from amino acid C226 up to the C-terminal end, the numbering being indicated according to EU index or equivalent in Kabat.

The term “scFc fragment” (“single chain Fc”) is intended to mean a single-chain Fc fragment obtained by genetic fusion of two Fc monomers linked by a polypeptide linker. The linker can in particular be -(GGGGS)n-, wherein n is an integer from 1 to 3. The scFc folds naturally into a functional dimeric Fc region. The scFc fragment preferably has the sequence SEQ ID No.: 42 (which corresponds to SEQ ID No.: 36 fused, in the C-terminal position, to -GGGGS-fused, in the C-terminal position, to SEQ ID No.: 37) optionally followed by a lysine. The fusion of the protein according to the invention to at least one wild-type immunoglobulin fragment (in particular an Fc or scFc fragment) in the C-terminal position makes it possible to improve the stability and retention of the protein in the organism, and thus its bioavailability; it also makes it possible to improve its half-life in the organism. In addition, it can make it possible to simplify the purification of the molecule obtained by targeting or by using the Fc fragment during one of the purification steps. Preferably, the wild-type Fc fragment is chosen from the sequence SEQ ID No.: 36 and the sequence SEQ ID No.: 37, optionally followed by a lysine in the C-terminal position (226 or 227 amino acids, respectively, for SEQ ID No.: 36, 231 or 232 amino acids, respectively for SEQ ID No.: 37). The Fc fragment corresponding to the sequence SEQ ID No.: 36 comprises the CH2 and CH3 constant domains of a wild-type IgG and the partial hinge region in the N-terminal position (DKTHTCPPCP SEQ ID No.: 38). The fragment corresponding to the sequence SEQ ID No.: 37 comprises the CH2 and CH3 constant domains of a wild-type IgG and the whole hinge region in the N-terminal position (sequence EPKSSDKTHTCPPCP, SEQ ID No.: 39, a variant of the natural sequence present on a wild-type IgG, of sequence EPKSCDKTHTCPPCP, SEQ ID No.: 81). Preferably, the protein according to the invention fused to a wild-type Fc fragment in the C-terminal position has the sequence SEQ ID No.: 40 optionally followed by a lysine in the C-terminal position. Its corresponding nucleic acid has the sequence SEQ ID No.: 41 optionally followed by a codon encoding a lysine in the C-terminal position. Alternatively and preferably, its nucleic acid has been obtained by gene synthesis with codon optimization for Homo sapiens and has the sequence SEQ ID No.: 82. Preferably, the protein according to the invention fused to a wild-type scFc fragment in the C-terminal position has the sequence SEQ ID No.: 43 optionally followed by a lysine in the C-terminal position. It correspondingly nucleic acid has a sequence SEQ ID No.: 44 optionally followed by a codon encoding a lysine in the C-terminal position.

The wild-type Fc fragment or the wild-type scFc fragment used according to the invention can be mutated according to the “knobs-into-holes” technology. This technology is described in Genentech application WO 96/27011: it consists in obtaining heterodimers, which comprise and pair preferably at the level of an antibody CH3 constant domain. These heterodimers, preferably 2 Fc fragments or one scFc fragment, comprise various point mutations, which induce a “knobs-into-holes” interface. A first mutation on the first monomer induces a knob, and a second mutation on the second monomer induces a hole, such that the heterodimer preferentially pairs.

Preferably, the first monomer (i.e. an Fc fragment or an Fc monomer of the scFc fragment) comprises the T366Y mutation, and the second monomer (i.e. an Fc fragment or an Fc monomer of the scFc fragment) comprises the Y407T mutation.

The sequences described in the present application can be summarized as follows:

| SEQ ID No.: | Protein |
|---|---|
| 1 | Human factor X heavy chain (306 amino acids), |
| | comprising the activation peptide |
| 2 | Human factor X signal peptide, propeptide and |
| | light chain (182 amino acids) |
| 3 | Heavy chain activation peptide (52 amino acids) |
| 4 | Human factor X prepropeptide (488 amino acids) |
| 5 | Human factor X light chain (142 amino acids) |
| 6 | Activated human factor X heavy chain (FXa) (254 |
| | amino acids) |
| 7 | Human factor X signal peptide (31 amino acids) |
| 8 | Human factor X propeptide (9 amino acids) |
| 9 | Human factor X propeptide variant (9 amino acids) |
| 10 | FX-WT (488 amino acids) |
| 11 | Mutated sequence SEQ ID No.: 1 comprising the |
| | insertion of DFLAEGLTPR between amino acids |
| | 52 and 53 (mutation according to the invention) |
| 12 | Anti-Gla aptamer |
| 13 | Thrombin propeptide (19 amino acids) |
| 14 | Factor VII propeptide version 1 (“FVIIv1”) (40 |
| | amino acids) |
| 15 | Factor VII propeptide version 2 (“FVIIv2”) (18 |
| | amino acids) |
| 16 | Protein C propeptide (24 amino acids) |
| 17 | FX-IIa (458 amino acids) |
| 18 | Human factor X signal peptide fused to the |
| | thrombin propeptide |
| 19 | Human factor X signal peptide fused to FVIIv1 |
| 20 | Human factor X signal peptide fused to FVIIv2 |
| 21 | Human factor X signal peptide fused to the protein |
| | C propeptide |
| 22 | Protein according to the invention (SEQ ID No.: 18 |
| | fused to SEQ ID No.: 17) |
| 23 | Protein according to the invention (SEQ ID No.: 19 |
| | fused to SEQ ID No.: 17) |
| 24 | Protein according to the invention (SEQ ID No.: 20 |
| | fused to SEQ ID No.: 17) |
| 25 | Protein according to the invention (SEQ ID No.: 21 |
| | fused to SEQ ID No.: 17) |
| 26 | Nucleic sequence encoding SEQ ID No.: 7 |
| 27 | Nucleic sequence encoding SEQ ID No.: 13 |
| 28 | Nucleic sequence encoding SEQ ID No.: 14 |
| 29 | Nucleic sequence encoding SEQ ID No.: 15 |
| 30 | Nucleic sequence encoding SEQ ID No.: 16 |
| 31 | Nucleic sequence encoding SEQ ID No.: 17 |
| 32 | Nucleic sequence encoding SEQ ID No.: 22 |
| 33 | Nucleic sequence encoding SEQ ID No.: 23 |
| 34 | Nucleic sequence encoding SEQ ID No.: 24 |
| 35 | Nucleic sequence encoding SEQ ID No.: 25 |
| 36 | Wild-type Fc fragment, optionally followed by a |
| | lysine |
| 37 | SEQ ID No.: 36 comprising the whole hinge region |
| | in the N-terminal position, optionally followed by a |
| | lysine |
| 38 | Partial hinge region in the N-terminal position |
| 39 | Whole hinge region in the N-terminal position |
| 40 | Protein SEQ ID No.: 23 fused to the wild-type Fc |
| | fragment SEQ ID No.: 36, optionally followed by a |
| | lysine |
| 41 | Nucleic acid encoding the protein SEQ ID No.: 40, |
| | optionally followed by a codon encoding a lysine |
| 42 | Wild-type scFc fragment, optionally followed by a |
| | lysine |
| 43 | Protein SEQ ID No.: 23 fused to the wild-type scFc |
| | fragment SEQ ID No.: 42, optionally followed by a |
| | lysine |
| 44 | Nucleic acid encoding the SEQ ID No.: 43 protein, |
| | optionally followed by a codon encoding a lysine |
| 45 | Wild-type human VKORC1 |
| 46 | Nucleic acid encoding the subunit SEQ ID No.: 45 |
| 47 | Prothrombin signal peptide |
| 48 | Factor VII signal peptide |
| 49 | Protein C signal peptide |
| 50 to 80 | Primers of the examples |
| 81 | Native whole hinge region in the N-terminal |
| | position |
| 82 | Optimized nucleic acid sequence encoding the |
| | protein SEQ ID No.: 40, optionally followed by a |
| | codon encoding a lysine |

Another subject of the invention is a nucleic acid (polynucleotide) encoding said protein. Preferably, the nucleic acid is chosen from the sequence SEQ ID Nos.: 32 to 35.

Another subject of the invention is an expression vector comprising said polynucleotide encoding said protein, or an expression cassette comprising said polynucleotide. According to the invention, the expression vectors suitable for use according to the invention may comprise at least one expression control element functionally bonded to the nucleic acid sequence. The expression control elements are inserted into the vector and make it possible to regulate the expression of the nucleic acid sequence. Examples of expression control elements include, in particular, lac systems, the lambda phage promoter, yeast promoters and viral promoters. Other functional elements can be incorporated, such as a leader sequence, stop codons, polyadenylation signals and sequences required for the subsequent transcription and translation of the nucleic acid sequence in the host system. It will be understood by those skilled in the art that the correct combination of the expression control elements depends on the host system chosen. It will also be understood that the expression vector must contain the additional elements required for the subsequent transfer of the expression vector containing the nucleic acid sequence into the host system and the subsequent replication of said vector therein.

Such vectors are easily constructed using conventional or commercially available methods.

Preferably, the expression vector used is a polycistronic vector comprising a polynucleotide encoding a protein according to the invention, a polynucleotide encoding the VKOR enzyme, preferably encoding the wild-type human vitamin K epoxide reductase complex subunit 1 (VKORC1), and optionally a polynucleotide encoding furin, and/or a polynucleotide encoding an Fc fragment in the context of the production of variants according to the invention, fused to an Fc fragment. The coexpression of furin makes it possible to optimize the natural cleavage inside the cell at the level of the natural cleavage sites present on factor X (RRKR). Preferably, the expression vector used is a bicistronic vector comprising a polynucleotide encoding a protein according to the invention, and a polynucleotide encoding the VKOR enzyme, preferably encoding the wild-type human vitamin K epoxide reductase complex subunit 1 (VKORC1).

The wild-type human vitamin K epoxide reductase complex subunit 1 (VKORC1) is the catalytic subunit of the complex; it is a protein of 163 amino acids. The sequence of this wild-type human subunit can be found in UniProt under accession number Q9BQB6 (SEQ ID No.: 45). The nucleic acid encoding this protein has the sequence SEQ ID No.: 46. This protein is present in the endoplasmic reticulum of cells. The polynucleotide encoding the VKOR enzyme can also be a polynucleotide encoding a mutated VKOR.

Preferably, alternatively, there are as many expression vectors used as there are polynucleotides to be expressed, one comprising a polynucleotide encoding a protein according to the invention, another comprising a polynucleotide encoding the abovementioned VKOR enzyme, optionally yet another comprising a polynucleotide encoding furin, and/or yet another comprising a polynucleotide encoding an Fc or scFc fragment in the context of the production of variants according to the invention, fused to an Fc or scFc fragment.

Another subject of the invention is a recombinant cell comprising an expression vector as described above, or a polynucleotide as described above. According to the invention, examples of host cells which can be used are eukaryotic cells, such as animal, plant, insect and yeast cells; and prokaryotic cells, such as E. coli. The means by which the vector carrying the gene can be introduced into the cells comprise in particular microinjection, electroporation, transduction or transfection using DEAE-dextran, lipofection, calcium phosphate or other procedures known to those skilled in the art. In one preferred embodiment, the expression vectors allowing expression in eukaryotic cells are used. Examples of such vectors comprise viral vectors, such as retroviruses, adenoviruses, herpes virus, vaccinia virus, smallpox virus, polio virus or lentiviruses, bacterial expression vectors or plasmids such as pcDNA5. The preferred eukaryotic cell lines comprise COS cells, CHO cells, HEK cells, in particular HEK293 (ATCC #CRL1573), YB2/0 cells, BHK cells, PerC6 cells, HeLa cells, NIH/3T3 cells, T2 cells, dendritic cells or monocytes. Preferably, the cells used are HEK cells. More preferably, the cells used are YB2/0 cells. More preferably, the cells used are CHO cells.

Another subject of the invention is a method for producing a protein according to the invention, said protein comprising a light chain (preferably SEQ ID No.: 5), comprising:
a) the expression of a polycistronic, preferably bicistronic, vector in a host cell, preferably an HEK cell, more preferably YB2/0, even more preferably a CHO cell, said vector comprising a polynucleotide encoding a protein according to the invention, and a polynucleotide encoding the VKOR enzyme, preferably encoding the wild-type human vitamin K epoxide reductase complex subunit 1 (VKORC1), preferably in the presence of vitamin K;
b) the culturing of said host cell;
c) the recovery of the cell supernatant;
d) optionally at least one of the steps chosen from:
   clarification of the supernatant, optionally followed by a filtration step,
   concentration of the supernatant,
   neutralization of the activated proteases by addition of protease inhibitors;
e) the purification of the protein according to the invention by passing the production supernatant obtained in c) or d) over a column of aptamers capable of binding to the Gla domain of factor X.

All culture conditions well known to those skilled in the art can be used for culturing the host cell in step b). For example, any production mode can be chosen, it being possible for the culturing to thus be carried out in batchwise, fedbatch, continued perfusion or XD process production mode, without being limited.

The concentration of the supernatant optionally carried out in step d) can be carried out by any well-known technique, such as by passing over concentration cassettes, by tangential filtration, or by using chromatography columns which make it possible to concentrate the product.

Another subject of the invention is a method for producing a protein according to the invention, said protein comprising a light chain (preferably SEQ ID No.: 5), comprising:
a) the expression of two expression vectors, one comprising a polynucleotide encoding a protein according to the invention, and the other comprising a polynucleotide encoding the abovementioned VKOR enzyme, in a host cell, preferably a CHO cell, preferably in the presence of vitamin K;
b) the culturing of said host cell;
c) the recovery of the cell supernatant;
d) optionally at least one of the steps chosen from:
   clarification of the supernatant, optionally followed by a filtration step,
   concentration of the supernatant,
   neutralization of the activated proteases by addition of protease inhibitors;
e) the purification of the protein according to the invention by passing the production supernatant obtained in c) or d) over a column of aptamers capable of binding to the Gla domain of factor X.

The two methods mentioned above advantageously make it possible to obtain factor X mutants according to the invention exhibiting a degree of gamma-carboxylation identical to that of plasma factor X, close to 100%.

The aptamers used in the methods described above are in particular those described in patent application WO 2011/012831. In particular, the aptamer used has the following sequence:

(SEQ ID No.: 12)

5′ CCACGACCTCGCACATGACTTGAAGTAAAACGCGAATTAC 3′.

Advantageously, this aptamer binds specifically to the biologically active forms of factor X. Thus, the methods for producing a protein according to the invention, comprising a purification step using a column of aptamers described above, make it possible to obtain biologically active forms of factor X.

The protein according to the invention can be produced in the milk of transgenic animals.

In this case, according to a first aspect, the expression of the polynucleotide encoding the protein according to the invention is controlled by a mammalian casein promoter or a mammalian whey promoter, said promoter not naturally controlling the transcription of said gene, and the polynucleotide also contains a protein secretion sequence. The secretion sequence comprises a secretion signal inserted between the gene and the promoter.

The transgenic animal used is capable not only of producing the desired protein, but also of transmitting this capacity to its progeny. The secretion of the protein in the milk facilities the purification and avoids the use of blood products. The animal can thus be chosen from mice, goats, does, ewes or cows.

The protein according to the invention can be used as a medicament. Consequently, the protein according to the invention can be introduced into a pharmaceutical composition. In particular, the protein according to the invention can be used for the treatment of coagulation disorders, in particular of hemorrhagic disorders.

The pharmaceutical composition of the invention can be combined with pharmaceutically acceptable excipients, and optionally sustained release matrices, such as biodegradable polymers, in order to form a therapeutic composition.

The pharmaceutical composition of the present invention can be administered orally, sublingually, subcutaneously, intramuscularly, intravenously, intra-arterially, intrathecally, intraocularly, intracerebrally, transdermally, locally or rectally. The active ingredient, alone or in combination with another active ingredient, can then be administered in unit administration form, as a mixture with conventional pharmaceutical carriers. Unit administration forms comprise oral forms, such as tablets, gel capsules, powders, granules and oral solutions or suspensions, sublingual and buccal administration forms, aerosols, subcutaneous implants, transdermal, topical, intraperitoneal, intramuscular, intravenous, subcutaneous and intrathecal administration forms, intranasal administration forms and rectal administration forms.

Preferably, the pharmaceutical composition contains a pharmaceutically acceptable vehicle for a formulation capable of being injected. This may involve in particular sterile isotonic formulae, saline solutions (with monosodium or disodium phosphate, sodium chloride, potassium chloride, calcium chloride or magnesium chloride and the like, or mixtures of such salts), or lyophilized compositions, which, when sterilized water or physiological saline is added, as appropriate, enable the constitution of injectable solutes.

The pharmaceutical forms suitable for injectable use comprise sterile aqueous solutions or dispersions, oily formulations, including sesame oil, peanut oil, and sterile powders for the extemporaneous preparation of sterile injectable solutions or of dispersions. In any event, the form must be sterile and must be fluid since it must be injected using a syringe. It must be stable under the manufacturing and storage conditions and must be preserved against the contaminating action of microorganisms, such as bacteria and fungi.

The dispersions according to the invention can be prepared in glycerol, liquid polyethylene glycols or mixtures thereof, or in oils. Under normal conditions of storage and use, these preparations contain a preservative for preventing microorganism growth.

The pharmaceutically acceptable vehicle may be a solvent or dispersion medium containing, for example, water, ethanol, a polyol (for example, glycerol, propylene glycol, polyethylene glycol, and the like), suitable mixtures thereof, and/or vegetable oils. Suitable fluidity may be maintained, for example, by using a surfactant, such as lecithin. The prevention of the action of microorganisms can be brought about by various antibacterial and antifungal agents, for example parabens, chlorobutanol, phenol, sorbic acid or else thimerosal. In many cases, it will be preferable to include isotonic agents, for example sugars or sodium chloride. The prolonged absorption of the injectable compositions can be brought about through the use in the compositions of absorption-delaying agents, for example aluminum monostearate or gelatin.

The sterile injectable solutions are prepared by incorporating the active substances in the required amount into the suitable solvent with several of the other ingredients listed above, where appropriate followed by filtration sterilization. As a general rule, the dispersions are prepared by incorporating various sterilized active agents into a sterile vehicle which contains the basic dispersion medium and the other ingredients required among those listed above. In the case of sterile powders for the preparation of sterile injectable solutions, the preferred preparation processes are vacuum-drying and lyophilization. During formulation, the solutions will be administered in a manner compatible with the dosage-regimen formulation and in a therapeutically effective amount. The formulations are easily administered in a variety of pharmaceutical forms, such as the injectable solutions described above, but drug-release capsules and the like can also be used. For parenteral administration in an aqueous solution for example, the solution must be suitably buffered and the liquid diluent must be made isotonic with a sufficient amount of saline solution or of glucose. These particular aqueous solutions are particularly suitable for intravenous, intramuscular, subcutaneous and intraperitoneal administration. In this regard, the sterile aqueous media which can be used are known to those skilled in the art. For example, a dose can be dissolved in 1 ml of isotonic NaCl solution and then added to 1000 ml of suitable liquid, or injected on the proposed site of the infusion. Certain dosage-regimen variations will necessarily have to be carried out according to the condition of the subject treated.

The pharmaceutical composition of the invention can be formulated in a therapeutic mixture comprising approximately 0.0001 to 1 0 milligrams, or approximately 0.001 to 0.1 milligrams, or approximately from 0.1 to 1.0 milligrams, or even approximately 10 milligrams per dose or more. Multiple doses can also be administered. The level of therapeutically effective dose specific for a particular patient will depend on a variety of factors, including the disorder which is treated and the seriousness of the disease, the activity of the specific compound used, the specific composition used, the age, bodyweight, general health, sex and diet of the patient, the time of the administration, the route of administration, the excretion rate of the specific compound used, the duration of the treatment, or else the medicaments used in parallel.

The protein according to the invention can also be used as a gene or cell therapy product.

To this effect, the present invention also relates to an expression vector comprising a polynucleotide encoding a protein according to the invention, said polynucleotide being as described above. This expression vector can be used as a medicament, preferably as a gene therapy medicament.

This expression vector can also be used as a cell therapy medicament: in this case, it is intended to be injected ex vivo into a sample of cells from a patient, before reinjection of said cells.

The following examples are given for the purpose of illustrating various embodiments of the invention.

### FIGURE LEGENDS

FIG. 1: primary structure of human factor X

FIG. 2: bicistronic vector OptiHEK-VKOR-FX-IIa

FIG. 3: final vector FVIIv1-psFX-IIa-F2

FIG. 4: evaluation of the level of gamma-carboxylation of the various FX variants

FIG. 5: evaluation of the FXs after purification

2 μg of product/lane were deposited.

A, SDS-PAGE of plasma FXs (lane 2), immunopurified CHO FX-IIa-F2 (lane 4) and aptamer-purified CHO FX-IIa-F2 (lane 3).

B, SDS-PAGE of plasma FXs (lane 2) and aptamer-purified HEK FX-IIa-F2 (lane 3). In lane 1: molecular weight or MW markers (the values in kDa are indicated on the left of the figure). NR: non-reduced products: DTT-R: products reduced with DTT.

FIG. 6: activation of variant FXs by the RVV-X fraction

Activated plasma FX (FXa) (●), plasma FX (▪), aptamer-purified FVIIv1-psFX-IIa-F2 from HEK (◯), aptamer-purified FVIIv1-psFX-IIa-F2-VKOR from HEK (□), aptamer-purified FVIIv1-psFX-IIa-F2 from HEK (Δ).

FIG. 7: activation of variant FXs by the FVIIa/Tissue Factor (TF) complex

Plasma FXa (●), plasma FX (▪), aptamer-purified FVIIv1-psFX-IIa-F2-VKOR from HEK (□), aptamer-purified FVIIv1-psFX-IIa-F2 from HEK (Δ).

FIG. 8: thrombin generation by the modified FXs in FVIII-deficient plasma

The thrombin generation was monitored over time in a normal plasma or a factor VIII-deficient plasma stimulated with 0.5 pM of tissue factor in the presence of phospholipids. Signal obtained with a normal plasma (●); signal obtained in FVIII deficient plasma (◯); the presence of 0.1 U/ml of recombinant FVIII (□); in the presence of 1 U/ml of recombinant FVIII (▪); in the presence of 10 μg/ml (Δ) or 20 μg/ml (▴) of aptamer-purified FVIIv1-psFX-IIa-F2 from HEK.

FIG. 9: evaluation of the FX-Fcs after purification

400 ng of product/lane were deposited.

SDS-PAGE of the plasma FXs (lanes 2/6), aptamer-purified FVIIv1-psFX-IIa-F2-Fc from CHO (lanes 3/4/7/8) and FVIIv1-psFX-IIa-F2-Fc from HEK (lanes 5/9). In lanes 1 and 10: molecular weight or MW markers (the values in kDa are indicated on the left of the figure). NR: non-reduced products; DTT-R: products reduced with DTT.

FIG. 10: evaluation of the binding of the FX-Fcs to phospholipids

Plasma FX (x), supernatant (◯) or aptamer-purified (●) FVIIv1-psFX-IIa-F2-Fc from YB2/0, supernatant (□) or aptamer-purified (▪) FVIIv1-psFX-IIa-F2-Fc from HEK, supernatant (Δ) or aptamer-purified (▴) FVIIvl-psFX-IIa-F2-Fc from CHO-F.

FIG. 11: activation of the variant FX-Fcs by thrombin

Plasma FX (x), aptamer-purified FVIIvl-psFX-IIa-F2-Fc from YB2/0 (●) aptamer-purified FVIIvl-psFX-IIa-F2-Fc from HEK (▪), aptamer-purified FVIIvl-psFX-IIa-F2-Fc from CHO-F (▴).

FIG. 12: thrombin generation by the modified FX-Fcs in FVIII-deficient plasma

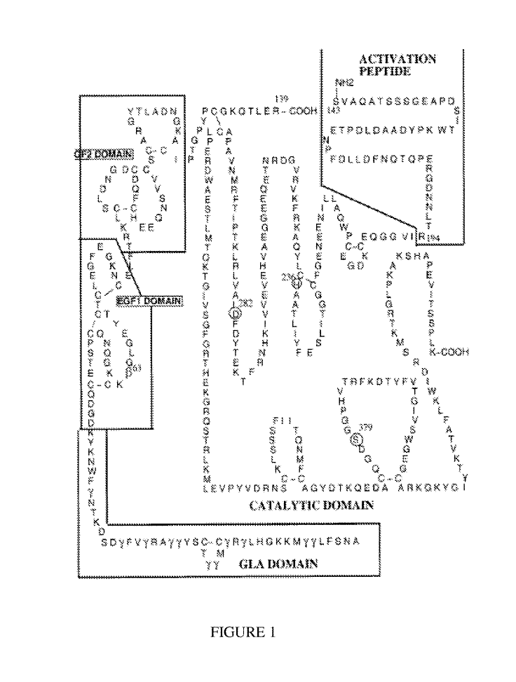

The thrombin generation was monitored over time in a normal or factor VIII-deficient plasma stimulated with 0.5 pM of tissue factor in the presence of phospholipids. Signal obtained with a normal factor (●); in the presence of 0.1 U/ml of recombinant FVIII (□); in the presence of 1 U/ml of recombinant FVIII (▪); in the presence of 4 μg/ml of aptamer-purified FVIIvl-psFX-IIa-F2-Fc from YB2/0 (Δ); in the presence of 4 μg/ml of aptamer-purified FVIIv 1-psFX-IIa-F2-Fc from HEK (▴); in the presence of 4 μg/ml of aptamer-purified FVIIv1-psFX-IIa-F2-Fc from CHO (♦).

### EXAMPLE 1

### Generation of the Expression Vectors Containing the Modified Propeptides

Construction of a Bicistronic Expression Vector Expressing a Modified FX and Human VKOR

A non-commercial expression vector (OptiCHO) was used to insert, by In Fusion ligation at the level of the Nhel-Swal restriction sites, a polynucleotide encoding the modified FX. Briefly, the OptiCHO expression vector was digested with the Nhel-Swal restriction enzymes and then gel-purified using the Nucleospin extract II kit (Macherey Nagel).

The modified FX polynucleotide was obtained by assembly PCR using, as template, a vector containing a polynucleotide encoding wild-type FX. The primers used are:

5′FXWT:

(SEQ ID No.: 50)

ACCAGCTGCTAGCAAGCTTGCCG
and
3′FX-2b:

(SEQ ID No.: 51)

GTCAGGCCCTCGCCAGGAAGTCCCTAGTCAGATTGTTATCGCCTCTTTC
AGGC for the first PCR,
and
5′FX-1b

(SEQ ID No.: 52)

AGGGCCTGACCCCTAGGATCGTGGGAGGACAGGAGTGCAAGGA
and
3′FX-SwaI

(SEQ ID No.: 53)

GAAACTATTTAAATGGATCCTCACTTGCCGTCAATCAGC
for the second PCR.

The fragment of interest obtained by PCR was then cloned by In Fusion ligation into the OptiCHO vector digested beforehand at the Nhel and Swal restriction sites.

The polynucleotide sequence encoding human VKOR was obtained by gene synthesis with codon optimization for Homo sapiens. It was extracted from a parental vector (OptiHEK-v3-VKOR) with the whole of the promoter unit (CMV enhancer, RSV promoter, polynucleotide, BGH poly A termination signal) by AscI-Spel digestion. It was introduced into the previously constructed vector by ligation at the same AscI-Spel restriction sites. The human VKOR contained a hexahistidine tag in the C-terminal position.

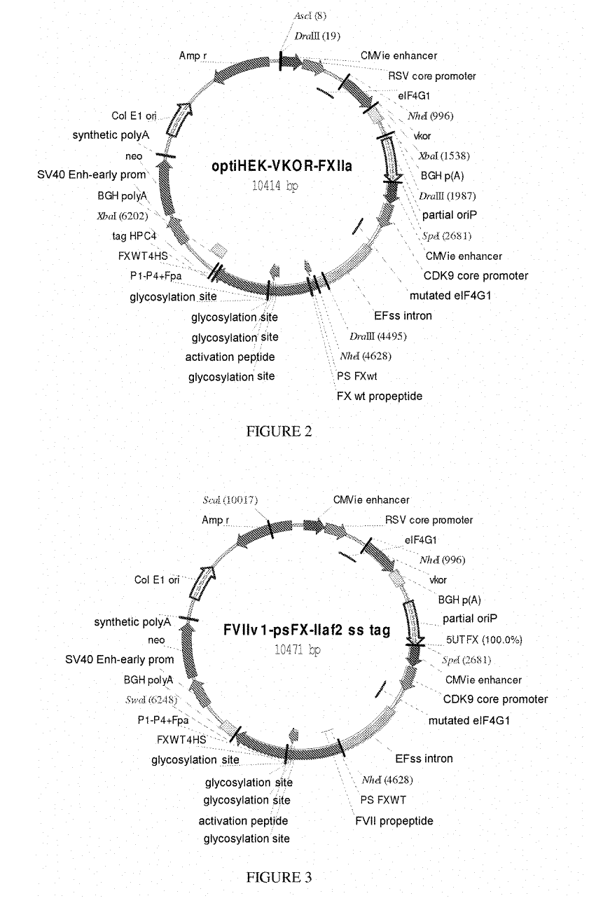

The final vector obtained containing 2 transcription units (bicistronic vector encoding, on the one hand, a modified FX and, on the other hand, the human VKOR) was called OptiHEK-VKOR-FXIIa (FIG. 2).

Construction of the Bicistronic Expression Vectors Expressing the Human VKOR and a Modified FX Containing a Signal Peptide and/or a Propeptide Different from That of wt FX

Preparation of the Final Expression Vector for the Ligation

The optiHEK-VKOR-FXIIa bicistronic vector which contains the transcription unit (UT) encoding, on the one hand, the modified FX and, on the other hand, the human VKOR was digested with the Spel-Swal restriction enzymes making it possible to obtain 2 fragments of 6904 and 3510 bp respectively. The 6904 bp fragment (digested vector) was gel-purified using the Nucleospin extract II kit (Macherey Nagel).

Preparation of a Promoter UT Allowing Expression of the Modified FX

The promoter unit of FX was amplified, by PCR, from a vector containing a polynucleotide encoding the modified FX, using the primers:

3UTFX:

(SEQ ID No.: 54)

GGTGGCGGCAAGCTTGCTAGC
and
5UTFX:

(SEQ ID No.: 55)

CCTTGGGCAATAAATACTAGTGGCGTTAC.

The amplicon obtained with the Kappa Hifi polymerase was then digested with the Nhel and Spel restriction enzymes so as to obtain a final fragment of 1983 bp. The fragment was purified on agarose gel and extracted using the Nucleospin extract II kit (Macherey Nagel).

Preparation of the Signal Peptide and Propeptide Inserts for the Expression of the Variant FXs

The signal peptide (PS) and the propeptide of FX-WT were replaced with those of prothrombin or of FVII isoform v1 (A) or of FVII isoform v2 (B) or of protein C. For this, the same strategy was applied each time (in the case of protein C, 3 primers were used to carry out the assembly PCR):
the signal peptide and the propeptide of interest were obtained by PCR from a vector containing the corresponding nucleotide sequences, using respectively the following primers:
prothrombin:

Primers 5PSth:

(SEQ ID No.: 56)

AAGCTTGCCGCCACCATGGCTCACGTCCGAGGGCTG
and
3PSth:

(SEQ ID No.: 57)

CTTCATTTCCTCCAGGAAAGAGTTGGCTCTCCGCACCCGCTGCAGC

FVII isoform v1 (A):

Primers 5PSFVII:

(SEQ ID No.: 58)

AAGCTTGCCGCCACCATGGTGTCTCAGGCTCTGCGGC
and
3PSFVII:

(SEQ ID No.: 59)

CAGGAAAGAGTTGGCCCTTCTCCTTCTATGCAGCACTCCATG

FVII isoform v2 (B):

Primers 5PSFVII:

(SEQ ID No.: 60)

AAGCTTGCCGCCACCATGGTGTCTCAGGCTCTGCGGC
and
3FVIIv2:

(SEQ ID No.: 61)

GTCACGAACACAGCAGCCAGACATCCCTGCAGTC

Protein C: primers

Protein 1:

(SEQ ID No.: 62)

AAGCTTGCCGCCACCATGTGGCAGCTGACCAGCCTGCTGCTGTTC
GTGGCCACATG,
Protein 2:

(SEQ ID No.: 63)

GAGCTGCTGAACACGCTATCCAGAGGGGCGGGTGTGCCAGAGAT
GCCCCATGTGGCCACG
Protein 3:

(SEQ ID No.: 64)

TTCAGCAGCTCTGAGCGGGCCCACCAGGTGCTGCGGATCAGAAAG
AGAGCCAACTCTTTC.

The sequence of the modified FX without the signal peptide and without the

FX-WT propeptide was obtained by PCR with the primers 5FX:

(SEQ ID No.: 65)

GCCAACTCTTTCCTGGAGGAAATGAAG
and
3FXIIa:

(SEQ ID No.: 66)

AGCTCTAGACAATTGATTTAAATGGATCCTCAC
(amplicon of 1142 bp).

An assembly PCR was carried out between these 2 PCR products.
A ligation by recombination (In Fusion ligation) was carried out between this assembly PCR product, the promoter UT and the digested final vector, prepared beforehand. The cloning efficiency was verified by PCR on colonies with the primers

5′EF1a:

(SEQ ID No.: 67)

GTGGAGACTGAAGTTAGGCCAG

and 2BGHpA and sequencing with the primers

5′EF1a:

(SEQ ID No.: 68)

GTGGAGACTGAAGTTAGGCCAG
and
5FXseq:

(SEQ ID No.: 69)

GGAGGCACTATCCTGAGCGAG.

The following bicistronic vectors were thus obtained:
proth-FX-IIa-F2: PS+prothrombin propeptide-modified FX+WT human VKOR
FVIIv1-FX-IIa-F2: PS+FVII isoform v1 propeptide-modified FX+WT human VKOR
FVIIv2-FX-IIa-F2: PS+FVII isoform v2 propeptide-modified FX+WT human VKOR
protc-FX-IIa-F2: PS+protein C propeptide-modified FX+WT human VKOR.

Replacement of the PS with the PS of FX-WT:

Using the 4 final vectors obtained, the following strategy was implemented in order to replace only the signal peptide with that of FX-WT:
1) The sequence corresponding to the PS of FX-WT was obtained from a vector containing the nucleotide sequence of the modified FX, by PCR with the primers PS1fxWT and PS2fxWT.
2) On each of the 4 final vectors, the sequence corresponding to that of the modified FX without signal peptide was obtained by PCR using the following primers:
proth-FX-IIa-F2:

primers 3FXIIA:

(SEQ ID No.: 70)

AGCTCTAGACAATTGATTTAAATGGATCCTCAC
ET PSWT-PRTH:

(SEQ ID No.: 71)

GACGGGAGCAGGCCCAGCATGTCTTCCTGGCACCACAG

FVIIv1-FX-IIa-F2:

primers 3FXIIA:

(SEQ ID No.: 72)

AGCTCTAGACAATTGATTTAAATGGATCCTCAC
ET PSWT-FVII v1:

(SEQ ID No.: 73)

CGGGAGCAGGCCGCTGGCGGCGTCGCTAAGGC

FVIIv2-FX-IIa-F2:

primers 3FXIIA:

(SEQ ID No.: 74)

AGCTCTAGACAATTGATTTAAATGGATCCTCAC
ET PSWT-FVII v2:

(SEQ ID No.: 75)

CGGGAGCAGGCCGCTGTGTTCGTGACCCAGGAAGAG

protc-FX-IIa-F2:

primers 3FXIIA:

(SEQ ID No.: 76)

AGCTCTAGACAATTGATTTAAATGGATCCTCAC
ET PSWT-PROT:

(SEQ ID No.: 77)

CGGGAGCAGGCCACACCCGCCCCTCTGGATAGCG

An assembly PCR was then carried out between the amplicon obtained in step 1 (PS of FX WT) and each of those obtained in step 2 (modified FX without signal peptide).

A ligation by recombination (In Fusion litigation) was carried out between these assembly PCR products, the promoter UT and the digested final vector, prepared beforehand.

The cloning efficiency was verified by PCR on colonies with the primers:

3) 5′EF1a:

(SEQ ID No.: 78)

GTGGAGACTGAAGTTAGGCCAG
and
4) 2BGHpA and sequencing with the primers
5′EF1A:

(SEQ ID No.: 79)

GTGGAGACTGAAGTTAGGCCAG
and
5FXSEQ:

(SEQ ID No.: 80)

GGAGGCACTATCCTGAGCGAG.

15

The following final bicistronic vectors were thus obtained:
proth-psFX-IIa-F2: PS FXwt+prothrombin propeptide-modified FX+WT human VKOR
FVIIv1-psFX-IIa-F2: PS FXwt+FVII isoform A propeptide-modified FX+WT human VKOR (FIG. 3)
FVIIv2-psFX-IIa-F2: PS FXwt+FVII isoform B propeptide-modified FX+WT human VKOR
protc-psFX-IIa-F2: PS FXwt+protein C propeptide-modified FX+WT human VKOR

The various sequences used in the examples are represented in the following table 1:

TABLE 1
Variant factor X sequences

| Sequence | Signal | | |
|---|---|---|---|
| Name | Peptide | Propeptide | FX-IIa sequence |
| FX-IIa | MGRPLHL | NNILARVRR | ANSFLEEMKKGHLERECMEETCSYEEARE |
| | VLLSASLA | (SEQ ID No: 9) | VFEDSDKTNEFWNKYKDGDQCETSPCQN |
| | GLLLLGES | | QGKCKDGLGEYTCTCLEGFEGKNCELFTR |
| | LFIRREQA | | KLCSLDNGDCDQFCHEEQNSVVCSCARGY |
| | (SEQ ID | | TLADNGKACIPTGPYPCGKQTLERRKRSV |
| | No: 7) | | AQATSSSGEAPDSITWKPYDAADLDPTENP |
| Proth- | MAHVRGL | QHVFLAPQQARS | FDLLDFNQTQPERGDNNLTRDFLAEGLTPR |
| FX-IIa | QLPGCLAL | LLQRVRR | IVGGQECKDGECPWQALLINEENEGFCGG |
| | AALCSLV | (SEQ ID No.: 13) | TILSEFYILTAAHCL YQAKRFKVRVGDRNT |
| | HS | | EQEEGGEAVHEVEVVIKHNRFTKETYDFDI |
| | (SEQ ID | | A VLRLKTPITFRMNV APACLPERDW AEST |
| | No.: 47) | | LMTQKTGIVSGFGRTHEKGRQSTRLKMLE |
| FVIIv1- | MVSQALR | AGGVAKASGGET | VPYVDRNSCKLSSSFIITQNMFCAGYDTKQ |
| FX-IIa | LLCLLLGL | RDMPWKPGPHR | EDACQGDSGGPHVTRFKDTYFVTGIVSWG |
| | QGCLA | VFVTQEEAHGVL | EGCARKGKYGIYTKVTAFLKWIDRSMKTR |
| | (SEQ ID | HRRRR | GLPKAKSHAPEVITSSPLK |
| | No.: 48) | (SEQ ID No.: 14) | (SEQ ID NO: 17) |
| FVIIv2- | MVSQALR | AVFVTQEEAHGV | |
| FX-IIa | LLCLLLGL | LHRRRR | |
| | QGCLA | (SEQ ID No.: 15) | |
| | (SEQ ID | | |
| | No.: 48) | | |
| ProC- | MWQLTSL | TPAPLDSVFSSSE | |
| FX-IIa | LLFVATW | RAHQVLRIRKR | |
| | GISG | (SEQ ID No.: 16) | |
| | (SEG ID | | |
| | No.: 49) | | |
| Proth- | MGRPLHL | QHVFLAPQQARS | ANSFLEEMKKGHLERECMEETCSYEEARE |
| psFX- | VLLSASLA | LLQRVRR | VFEDSDKTNEFWNKYKDGDQCETSPCQN |
| IIa* | GLLLLGES | (SEQ ID No.: 13) | QGKCKDGLGEYTCTCLEGFEGKNCELFTR |
| (SEQ ID | LFIRREQA | | KLCSLDNGDCDQFCHEEQNSVVCSCARGY |
| No.: 21) | (SEQ ID | | TLADNGKACIPTGPYPCGKQTLERRKRSV |
| FVIIv1- | No: 7) | AGGVAKASGGET | AQATSSSGEAPDSITWKPYDAADLDPTENP |
| psFX- | | RDMPWKPGPHR | FDLLDFNQTQPERGDNNLTRDFLAEGLTPR |
| IIa* | | VFVTQEEAHGVL | IVGGQECKDGECPWQALLINEENEGFCGG |
| (SEQ ID | | HRRRR | TILSEFYILTAAHCL YQAKRFKVRVGDRNT |
| No.: 22) | | (SEQ ID No.: 14) | EQEEGGEAVHEVEVVIKHNRFTKETYDFDI |
| FVIIv2- | | AVFVTQEEAHGV | A VEREKTPITFRMNV APACLPERDW AEST |
| psFX- | | LHRRRR | LMTQKTGIVSGFGRTHEKGRQSTRLKMLE |
| IIa* | | (SEQ ID No.: 15) | VPYVDRNSCKLSSSFIITQNMFCAGYDTKQ |
| (SEQ ID | | | EDACQGDSGGPHVTRFKDTYFVTGIVSWG |
| No.: 23) | | | EGCARKGKYGIYTKVTAFLKWIDRSMKTR |
| ProtC- | | TPAPLDSVFSSSE | GLPKAKSHAPEVITSSPLK |
| psFX- | | RAHQVLRIRKR | (SEQ ID NO: 17) |
| IIa* | | (SEQ ID No.: 16) | |
| (SEQ ID | | | |
| No.: 24) | | | |

*mutant according to the invention

### EXAMPLE 2

### Production of the FXs Containing Modified Propeptides in the HEK 293 Freestyle Production Line

1. Reagents

Freestyle™ F17 culture medium

L-glutamine

HEK cell transfection medium: Opti-MEM

Vitamin K1

2. Protocol

The wild-type factor X and the modified FXs were produced in HEK-293-Freestyle eukaryotic cells (HEK 293F) in transient expression.

The HEK 293F cells were cultured in F17 medium, supplemented with 8 Mm of L-glutamine, under stirred conditions at 135 rpm in a controlled atmosphere (8% CO₂) at 37° C. On the day before the day of transfection, the cells were seeded at a density of 7×10⁵ cells/ml. On the day of transfection, the DNA (30 μg) and 16 μl of transfection agent (TA) were preincubated separately in Opti-MEM medium for 5 minutes and then mixed and incubated for 20 minutes so as to allow the formation of the DNA/TA complex. The whole mixture was added to a cell preparation of 1×10⁶ cells/ml in a volume of 30 ml.

In the case of cotransfections, the 2 vectors were added at various ratios so as to obtain a total amount of DNA of 20-30 μg Immediately after the transfection, the vitamin K1 (5 μg/ml) was added to the medium. The degrees of transfection were evaluated the day after transfection using a control plasmid expressing GFP (Green Fluorescent Protein). The productions were carried out in “batchwise” mode for 7 days. At the end of production, the cells and the supernatant were separated by centrifugation. The cells were eliminated and the supernatant was harvested, supplemented with 2 mM PMSF and 10 mM benzamidine, filtered through 0.22 μm, concentrated 10× and then frozen.

### EXAMPLE 3

### Production of the FXs Containing Modified Propeptides in the CHO-S Production Line

1. Reagents

ProCHO4 culture medium

L-glutamine

CHO-S cell transfection medium: Opti-Pro SFM

Vitamin K1

2. Protocol

The wild-type factor X and the modified FXs were produced in CHO-S eukaryotic cells (Invitrogen) in transient expression.

The CHO-S cells were cultured in proCHO4 medium, supplemented with 4 mM of L-glutamine, under stirred conditions at 135 rpm in a controlled atmosphere (8% CO₂) at 37° C. On the day before the day of transfection, the cells were seeded at a density of 6×10⁵ cells/ml.

On the day of transfection, the DNA (37.5 μg) and 37.5 μl of transfection agent (TA) were preincubated separately in Opti-Pro SFM medium for 5 minutes and then mixed and incubated for 20 minutes so as to allow the formation of the DNA/TA complex. The whole mixture was added to a cell preparation of 1×10⁶ cells/ml in a volume of 30 ml.

In the case of cotransfections, the 2 vectors were added at various ratios so as to obtain a total amount of DNA of 20-45 μg Immediately after the transfection, the vitamin K1 (5 μg/ml) was added to the medium. The degrees of transfection were evaluated the day after transfection using a control plasmid expressing GFP. The productions were carried out in “batchwise” mode for 7 days. At the end of production, the cells and the supernatant were separated by centrifugation. The cells were eliminated and the supernatant was harvested, supplemented with 2 mM PMSF and 10 mM benzamidine, filtered through 0.22 μm, concentrated 10× and then frozen.

### EXAMPLE 4

### Quantification of the Gamma-Carboxylation of the Factors X Produced

1—Experimental Protocol: Measurement of the Factor X Concentration

The factor X concentration was measured by means of the Zymutest factor X commercial ELISA (Hyphen BioMed ref RK033A) according to the recommendations of the producer. The concentrations were measured in duplicate using antigen values located in the linear detection zone of the assay. In order to be sure that the mutations introduced do not disrupt the concentration measurement, the FXs were deposited in identical amount and revealed by immunoblotting with a polyclonal antibody different than that used in ELISA (anti-human-FX polyclonal antibody (CRYOPEP cat No. PAHFX-S)) or by staining after SDS-PAGE (data not shown).

The concentrations of the variant FXs present in the supernatants of the transfected HEK cells were measured in order to deposit the same amount of FX on the anti-Gla ELISA.

2—Experimental Protocol: Measurement of the Degree of Gamma-Carboxylation

The degree of gamma-carboxylation was measured by means of an ELISA established in the laboratory which uses the revealing antibody of the Zymutest factor X ELISA assay kit (Hyphen) and the anti-Gla antibody (Seikisui) as capture antibody.

The anti-Gla antibody (200 μl at 5 μg/ml) was incubated overnight at ambient temperature (AT). After incubation, the plate was saturated with PBS+1% BSA (250 μl/well) for 2 h at AT. After washing, 200 μl of sample at 0.2 μg/ml or of standards (consisting of the mixture at various ratios of plasma FX and of factor X produced in CHO (non-gamma-carboxylated)) were deposited for 2 h at AT. After washing operations, the peroxidase-coupled anti-FX antibody (200 μl of the Zymutest kit) was diluted in the buffer provided and incubated for 1 h at AT. After washing operations, the revealing was carried out by adding 200 μl of TMB for 8 minutes. The revealing was stopped with 50 μl of sulfuric acid at 0.45 M and the optical density was read at 450 nm.

3—Results

HEK cells naturally produce ectopic FX in non-gamma-carboxylated form (not shown). In order to advantageously increase the degree of gamma-carboxylation, the FX was cotransfected in the presence of VKOR. This cotransfection can be carried out either by treating the cells with two vectors (Opti-HEK-FX-IIa-F2) or using a bicistronic vector carrying the two cDNAs (Opti-HEK-VKOR-IIa). In both cases, the degree of gamma-carboxylation was identical at 11.55% and 10.20% of the plasma-FX respectively (table 2). Complete replacement of the FX signal peptide and of the propeptide with those of FVII (v1 and v2), of prothrombin or of protein C did not significantly increase the degree of gamma-carboxylation (6.7 to 24.5%). However, the combination with FVIIv1 (FVIIv1-FX-IIa-F2) is the most efficient of the 4 at 24.5%.

Chimeric constructs were then constructed by retaining the FX signal peptide and inserting the propeptides previously used. The new constructs thus generated prove, surprisingly, to be advantageous in terms of gamma-carboxylation, especially that with the FVIIv1 propeptide (FVIIv1-psFX-IIa-F2) for which a degree of 52% was observed.

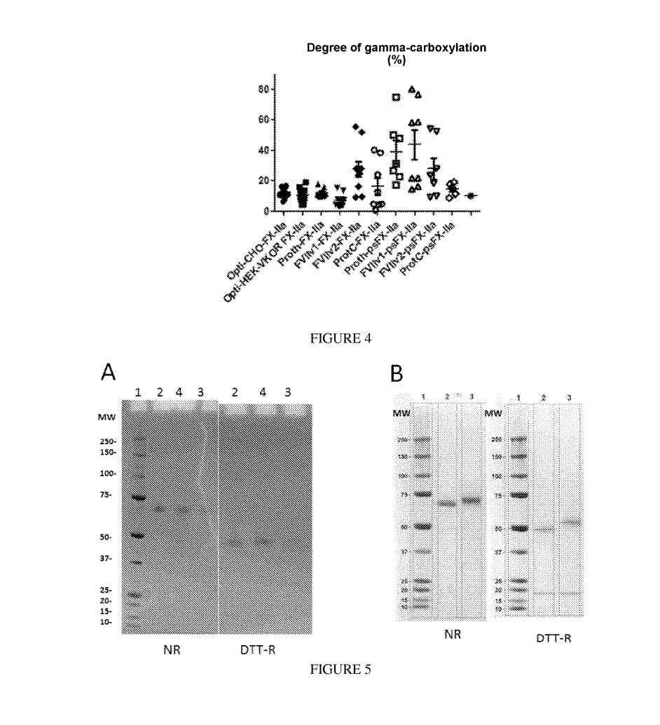

Thus, the latter construct makes it possible to increase the degree of gamma-carboxylation 4.7 fold. All of the individual measurements have been presented in FIG. 4 which shows the superiority of the use of the FX combination and of the FVIIv1 propeptide.

TABLE 2
evaluation of the gamma-carboxylation of the various mutants

| | | | Amount of production | | | |
|---|---|---|---|---|---|---|
| | % GLA | | (μg/ml) | | % Yield | |
| Opti-CHO-FX-IIa-F2 | 11.115 | [16.97-6.4984] | 0.647 | [0.45-0.82] | 83.790 | [96.95-62.85] |
| Opti-HEK-VKOR-IIa | 10.2 | [19.19-4.2627] | 0.375 | [0.18-0.71] | 71.677 | [46.7-86.899] |
| Proth-FX-IIa-F2 | 12.21 | [9.81-17.64] | 0.243 | [0.17-0.3] | 83.653 | [45.09-144.65] |
| FVIIv1-FX-IIa-F2 | 6.66 | [3.2707-15.54] | 0.151 | [0.09-0.21] | 74.549 | [45.54-97.48] |
| FVIIv2-FX-IIa-F2 | 24.47 | [9.4374-55.58] | 0.269 | [0.15-0.45] | 77.163 | [45.83-90.61] |
| ProtC-FX-IIa-F2 | 14.71 | [0.54-40.2] | 0.219 | [0.17-0.29] | 87.586 | [66.90-27.3] |
| Proth-psFX-IIa-F2 | 44.8 | [17.53-75.035] | 0.388 | [0.23-0.5] | 75.752 | [60.62-113.24] |
| FVIIv1-psFX-IIa-F2 | 52.03 | [14.79-80.209] | 0.513 | [0.35-0.74] | 83.133 | [76.22-87.17] |
| FVIIv2-psFX-IIa-F2 | 23.95 | [9.213-54.43] | 0.519 | [0.22-0.71] | 79.476 | [67.54-113.43] |
| ProtC-psFX-IIa-F2 | 13.83 | [9.05-18.98] | 0.413 | [0.37-0.46] | 78.863 | [57.72-150.54] |

### EXAMPLE 5

### Purification of the FXs Containing Modified Propeptides on a Column of Aptamers Capable of Binding the Gla Domain of Factor X

1. Protocol

The concentrated culture supernatant from HEK or CHO was thawed at 37° C. It was then diluted to ½ in equilibration buffer (50 mM Tris HCl, 10 mM CaCl₂, pH 7.5) and then purified on an anti-Gla aptamer column which was pre-equilibrated in the same buffer. The column was washed with 12 column volumes of equilibration buffer. The FX was then eluted with a 50 mM Tris HCl, 10 mM EDTA buffer, pH 7.5. The column was placed again in equilibration buffer (25 column volumes) before storage at 4° C. The FX was treated with 2 mM of PMSF, concentrated, and stored at —80° C.

2. Results

The FX-FIIa-F2s produced from CHO or HEK were purified on an aptamer recognizing the gamma-carboxylated domain. The CHO product was purified according to a conventional immunopurification protocol or by aptamer-purification. The purified products were controlled by SDS-(4-10%)PAGE (FIG. 5A). The two recombinant products showed a similar profile following separation in acrylamide with or without DTT reduction (FIG. 5A, lanes 4 and 3). When non-reduced, the products appeared in the form of a single band at approximately 60-65 kDA. Their migration was slightly slower than that of the plasma FX (FIG. 5A, lane 2) since the products have an additional 10 amino acids. The reduction of the products completely separates the heavy chain (48 kDa) from the light chain (17 kDa). The recombinant FXs showed a similar profile regardless of their purification mode. The light chain of the three purified factors X migrated in the same way, as expected.

The aptamer-purified HEK product was compared with the plasma FX (FIG. 5B). The product was pure to a level of homogeneity and appeared in the form of a single band migrating at a molecular weight slightly above that of the plasma FX as previously seen (FIG. 5B, lane 3). The reduction of the product shows that the difference in migration is carried by the heavy chain.

These data show that the aptamer purification, for example of FX-IIa-F2, makes it possible to obtain a product that is pure to a level of homogeneity after a single purification step.

### EXAMPLE 6

### Measurement of the Activation of the Variant Factors X Produced in HEK by the RVV-X Venom Fraction

1. Experimental Protocol

The activation of the variant FXs produced by the HEK cells was measured following the incubation of the aptamer-purified factors X in the presence of the Russell's viper venom anti-factor X fraction (RVV-X). The control activated factor X, the venom X fraction (RVV-X) and the pNAPEP 1065 substrate were commercially available (e.g. Haematologic Technologies).

The activation was studied at 37° C. in the following buffer: 25 mM HEPES, pH 7.4, 0.175 M NaCl, 5 mM CaCl₂, 5 mg/ml BSA. For concentrations of 0 to 100 nM of FX, a concentration of 200 mU/ml of RVV-X was used. After incubation for 5 min, the reaction was stopped in the 50 mM Tris buffer, pH 8.8, containing 0.475 M NaCl, 9 mM EDTA. The amount of FXa generated was monitored by measuring the rate of hydrolysis of the pNAPEP 1065 substrate (250 μM) at 405 nm.

2. Results

The purified factor X variants were incubated with the RVV-X. The generation of FXa was measured following this treatment starting from various FX concentrations. The presence of FXa was quantified by means of the rate of appearance of the pNAPEP 1065 product in solution (in mODU/min). This generation is a reflection of the recognition and of the cleavage of the FXs by the RVV-X and also of the capacity of the FXa generated to recognize the FX substrate. The mean rates of appearance was determined for the various initial concentrations of FX and this value was related to the percentage of the FX-WT value.

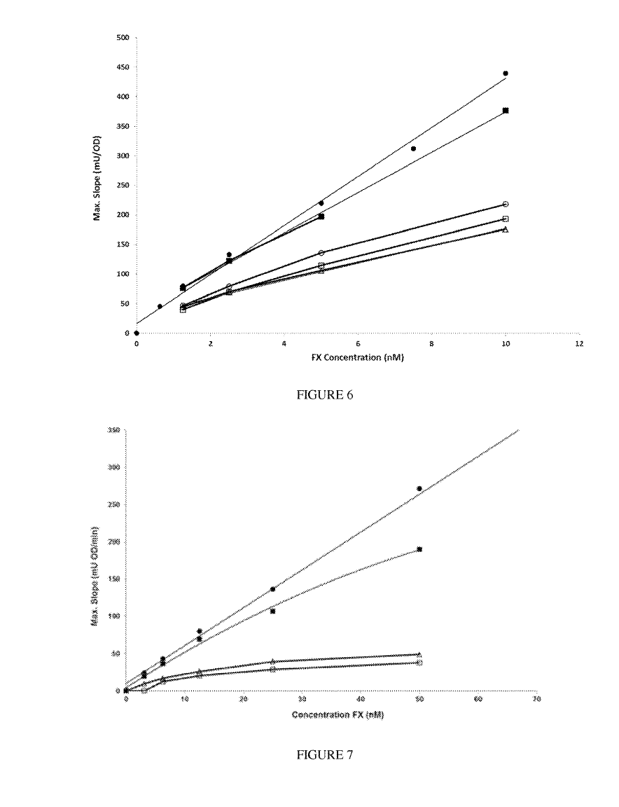

The controls of the FX already activated and of the FX treated with the RVV-X both gave a positive signal of the same order of magnitude (FIG. 6). The FXa control was considered to be 100%. The two variant factor X constructs were approximately 60% activated relative to the FXa (54% for Opti-HEK-VKOR-IIa and 63% for FVIIv1-psFX-IIa-F2). This result was not surprising since the activation by RVV-X is not sensitive to the degree of gamma-carboxylation. This result advantageously showed, on the other hand, that the modification of the propeptide does not lead to a loss of the chromogenic activity of FXa.

### EXAMPLE 7

### Measurement of the Activation of the Variant Factors X Produced in HEK by the Factor VIIa/Tissue Factor (TF) Complex

1. Experimental Protocol

The activation of the variant FXs produced by the HEK cells was measured following incubation of the aptamer-purified product in the presence of 50 pM of FVIIa and of tissue factor. In a flat-bottomed plate, the FVIIa (100 μl at 100 pM)-TF complex was added to various dilutions of FX (100 μ). After 10 min the mixture (20μ1) was removed and deposited in 180 μl of STOP buffer (50 mM Tris, 9 mM EDTA, 475 mM NaCl, pH 8.8). The PNAPEP substrate diluted to ½ WFI water (50 μl) was added and an immediate reading in kinetic mode was carried out every 25 seconds for 10 mM at 405 nm.

2. Results

The controls represented by the already activated plasma FX and the plasma FX treated with the FVIIa/FT both gave a positive signal of the same order of magnitude (FIG. 7). The plasma FXa control was considered to be 100%. The two variant factor X constructs were 27% activated relative to the FXa for Opti-HEK-VKOR-IIa and 36% activated relative to the FXa for FVIIv1-psFX-IIa-F2. This activity is sensitive to the degree of gamma-carboxylation. The modification of the propeptide allowed FVIIv1-psFX-IIa-F2 to have an activity of 142% of that of the molecule containing the wild-type propeptide. These results indicate that the increase in the degree of gamma-carboxylation makes it possible to increase the procoagulant activity of the variant FXa relative to the control molecule.

### EXAMPLE 8

### Measurement in Terms of Thrombin Generation Time (TGT) of the Procoagulant Capacity of the Variant Factors X: Activation of the Extrinsic Coagulation Pathway (TF 1 pM/PL 4 μM) in FVIII-Deficient Plasma

1. Experimental Protocol

1.1. Reagents

Thrombin calibrator, PPP reagent low, CK-Prest, FluCa Kit (Fluo-buffer+Fluo-substrate) and the PNP were commercially available, for example from Stago. The FVIII-deficient plasma (e.g. Siemens Healthcare) and the control recombinant human factor VIII come from Baxter (Advate).

1.2. Protocol

The thrombin generation test consists in activating the coagulation ex vivo using a mixture of tissue factor and of phospholipids (activation of the extrinsic coagulation pathway) and in then measuring the concentration of thrombin generated over time.

The thrombin generation tests were carried out on 80 μl of a plasma pool containing purified product or the controls, in the presence of 20 μl of PPP reagent containing a final concentration of 1 pM of tissue factor (TF) and 4 μM of phospholipids (PL). Various plasmas can be used: normal plasma, factor X-deficient plasma, factor VIII-deficient plasma, factor IX-deficient plasma or factor XI-deficient plasma.

The reaction was initiated by adding 20 μl of FluCa Kit (substrate+CaCl₂) which constitutes the beginning of the measurement of the appearance of thrombin. The appearance of fluorescence was measured on a Fluoroskan Ascent fluorometer (ThermoLabsystems) at an excitation wavelength of 390 nm and at an emission wavelength of 460 nm. The thrombinograms (curves representing the fluorescence intensity as a function of time) were then analyzed by means of the Thrombinoscope™ software which converts the fluorescence value into nM of thrombin by comparative calculation.

2. Results

The Unicalibrator plasmas, and also the FVIII-deficient plasmas reconstituted by 0, 0.1 or 1 U/ml of recombinant FVIII, were used as controls. The aptamer-purified FVIIv1-psFX-IIa-F2 was used at 10 and 20 μg/ml.

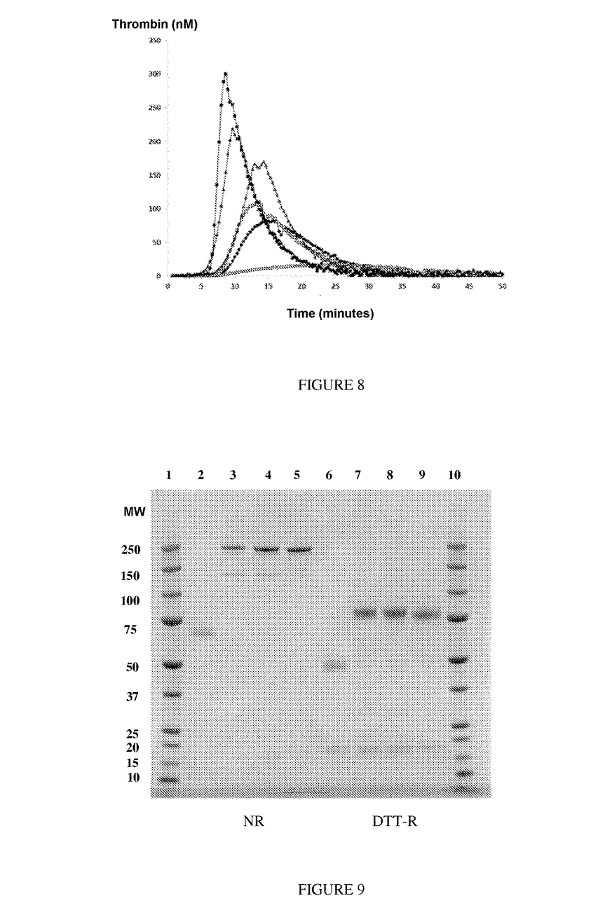

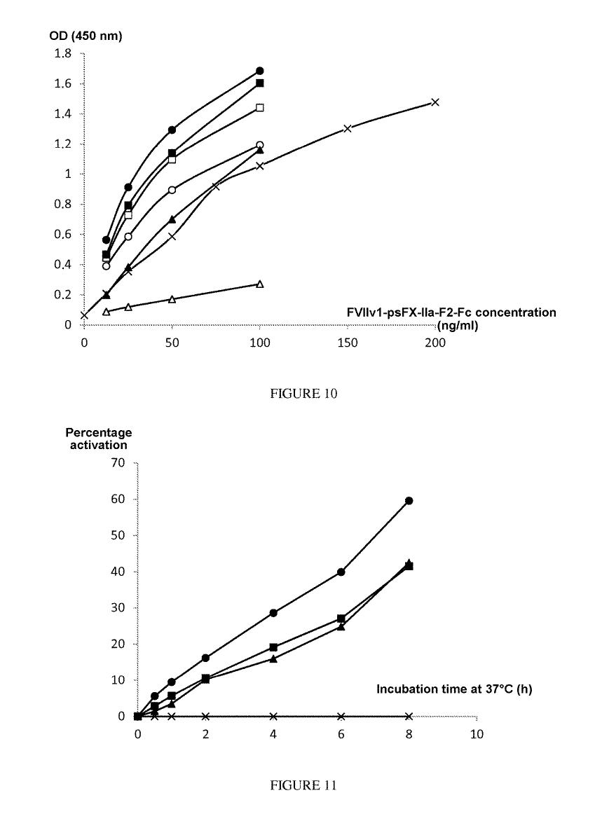

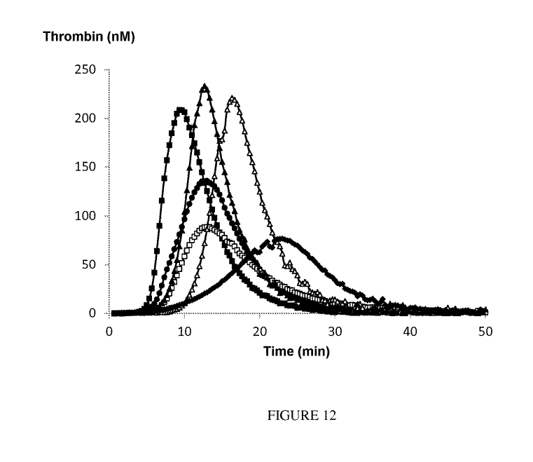

As expected, following the activation of coagulation by tissue factor, the FVIII-deficient plasma gave the weakest signal, corresponding to the background noise of the experiment (FIG. 8). The Unicalibrator plasma gave a weaker signal than the FVIII-deficient plasma reconstituted with the concentrations of FVIII (0.1 or 1 U/ml).

The variant FX having a modified propeptide has the capacity to correct an FVIII-deficient plasma as efficiently as FVIII. A dose-dependent response was observed with a lag time which shortens when the dose increases and an amplitude which increases. However, the amplitude of the signal did not completely reach the reconstitution with 1 U/ml of FVIII, but it was much greater than that of a normal plasma. Consequently, the increase in gamma-carboxylation obtained for a variant factor X according to the invention, advantageously coupled to an aptamer purification, made it possible to obtain a perfectly active variant factor X which efficiently replaces FVIII.

### EXAMPLE 9

### Production of the FX-Fcs Containing a Modified Propeptide in the YB2/0 Production Line

1. Reagents

Culture medium: EMabpro 1

L-glutamine 200 mM

50 mg/ml of Geneticin

LS100

Vitamin K1

2. Protocol

The modified FX FVIIv1-psFX-IIa-F2-Fc was cloned into a bicistronic vector optimized for expression in the YB2/0 line, into which a nucleic sequence encoding human furin had previously been introduced at the level of the second transcription unit. The amount of vector required for the transfection was then prepared and linearized at the EcoRV restriction site.

After centrifugation, the YB2/0 cells were taken up in a volume which makes it possible to obtain a cell density of 1×10⁷ cells/ml. The transfection was carried out by electroporation using a specific kit (ref: EB110, Ozyme) at 5×10⁶ cells/ml in the presence of 61.7 μg of bicistronic vector containing the FVIIv1-psFX-IIa-F2-Fc sequence and the human furin sequence. After transfection, the cells were resuspended in 75 cm² flasks. A selection pressure was added three days after transfection, by adding G418 at 0.6 g/l. The selection pressure was maintained for 14 days, then the cells were frozen. FVIIv1-psFX-IIA-F2-Fc molecule productions were launched by seeding the selected YB2/0 cells at a density of 3×10⁵ cells/ml in EMabprol medium containing 4 mM of glutamine For the production, a “fedbatch” mode was applied for 12 days, with glucose and glutamine being added as a function of the previously determined cell density.

At the end of production, the cells and the supernatant were separated by centrifugation. The cells were eliminated and the supernatant was harvested, supplemented with 2 mM PMSF and 10 mM benzamidine, concentrated 5×, filtered through 0.22 μm, then frozen.

### EXAMPLE 10

### Purification of the FX-Fcs Containing Modified Propeptides on a Column of Aptamers Capable of Binding the Gla Domain of Factor X

1. Protocol

The FVIIv1-psFX-IIa-F2-Fc was produced in HEK293F, CHO-S and YB2/0 as described in examples 2, 3 and 9.

The concentrated culture supernatant from HEK, YB2/0 or CHO-S was thawed at 37° C. and then filtered on a Nalgene 0.2 μm unit (aPES). For one volume of supernatant after filtration, one volume of 50 mM Tris-HCl buffer, pH 7.5, was added. QAE Sephadex A50 gel (0.25% weight/vol; GE Healthcare) was added and the whole mixture was then stirred for one hour at+4° C. The gel was loaded into a column body and washed with the equilibration buffer, and the molecules of interest were eluted with a 50 mM Tris-HCl buffer, pH 7.5, containing 500 mM NaCl. The eluent was then frozen at −80° C. before aptamer purification.

The thawed eluent was diluted to ½ in equilibration buffer (50 mM Tris HCl, 10 mM CaCl₂, pH 7.5) then purified on an anti-Gla aptamer column which had been pre-equilibrated in the same buffer. The column was washed with 12 column volumes of equilibration buffer. The FVIIv1-psFX-IIa-F2-Fc was then eluted with a 50 mM Tris HCl buffer containing 10 mM EDTA, pH 7.5. The column was placed again in equilibration buffer (25 column volumes+0.01% sodium azide) before storage at 4° C. The FVIIv1-psFX-IIa-F2-Fc was treated with 0.01 mM of the GGACK inhibitor (Cryopep), dialyzed against 0.9% NaCl, concentrated, and stored at −80° C.

2. Results

The FVIIv1-psFX-IIa-F2-Fcs produced from CHO-S, YB2/0 or HEK were purified on an aptamer recognizing the gamma-carboxylated domain The purified products from CHO-S or HEK were controlled by SDS-(4-10%)PAGE (FIG. 9). They showed a similar profile following the separation in acrylamide with or without DTT reduction (FIG. 9, lanes 3-5; 7-9). When not reduced, the proteins appeared in the form of a major band at approximately 250 kDa migrating very differently than that of the plasma FX (67 kDa; FIG. 9, lane 1) since the products are grafted to an Fc fragment. A minor band at 135 kDa was also detected only in the products from CHO. The reduction of the products completely separates the Fc-grafted heavy chain (81 kDa) from the light chain (17 kDa). The variant FX-Fcs showed a similar profile regardless of their mode of production. The light chain of the three purified factors X migrated in the same way, as expected, with however a greater heterogeneity in the product from CHO. It should be noted that a profile similar to that of HEK was obtained with the product from YB2/0 (not shown).

These data show that the aptamer purification, for example of the FVIIv1-psFX-IIa-F2-Fc, makes it possible to obtain a product pure to a level of homogeneity after a single purification step, even if this material is produced from various cell lines. The presence of a modified propeptide does not affect the capacity of the product to be purified by this method.

### EXAMPLE 11

### Phospholipid-Binding of the FX-Fcs Produced in Various Cell Lines

1. Protocol

The phospholipids were diluted to 12.5 μM in absolute ethanol and then loaded into 96-well plates. They were incubated overnight at ambient temperature without a lid. The wells were then saturated for 2 h with 50 mM Tris buffer containing 150 mM NaCl, 10 mM CaCl₂, 1% BSA, pH 7.5. After saturation, the wells were washed 5 times with the washing/diluting buffer (50 mM Tris, 150 mM NaCl, 10 mM CaCl₂, 0.1% BSA, pH 7.5) and then the samples were deposited at various dilutions and incubated for 2 h at ambient temperature. They were then washed 5 times before incubation with the peroxidase-coupled anti-FX antibody. The wells were washed 5 times before revealing: 3 minutes at ambient temperature with TMB (Zymutest FX kit). The reaction was stopped by adding 50 μl of 0.45 M sulfuric acid. The ODs at 450 nm were read after having slightly shaken the plate.

2. Results

The control plasma factor X (x) binds as expected to the phospholipids as a function of the starting concentration. The signal tends toward saturation. The non-purified FVIIv1-psFX-IIa-F2-Fcs present in the supernatants from CHO-S, HEK293 and YB2/0 and the same products which have been aptamer purified were evaluated. All the non-purified forms in supernatant bound less well to the phospholipids than the aptamer-purified forms. This difference in signal can originate either from the presence of molecules which inhibit or compete for the binding in the supernatants or, most probably, the supernatants contained a fraction of the weakly gamma-carboxylated product and they therefore bound less well to the phospholipids. Passing over the aptamer purification column increased to a variable extent the capacities of the three products to bind to the phospholipids: the product from CHO is the one for which the signal was the most improved, then followed by the YB2/0 product and then the HEK293 product. After aptamer purification, all the products bound to the phospholipids giving a signal at least as strong as that of the plasma FX or even stronger for the HEK293 and YB2/0 products.

These data show that the FX-IIa-F2-Fcs produced in the presence of a modified propeptide (e.g. FVIIv1-psFX-IIa-F2-Fc) have an excellent capacity to bind to the phospholipids, which binding is known to be mediated by the various gamma-carboxylation sites.

### EXAMPLE 12

### Activation of the FX-Fcs Produced in Various Cell Lines by Thrombin

1. Protocol

The experiments and the dilutions were performed in the following reaction buffer: 25 mM Hepes, 175 mM NaCl, 5 mg/ml BSA, 5 mM CaCl₂, pH 7.4. The standard range was prepared as follows: in a flat-bottomed plate, 100 μl of r-hirudin at 50 nM+100 μl of each dilution of FXa+50 μl of PNAPEP substrate diluted to ½ in WFI water. An immediate reading in kinetic mode was taken every 25 seconds for 10 min at 405 nm. The assays were carried out by adding 100 μl of sample at 200 nM, 100 μl of thrombin at 20 nM final concentrations 100 nM FX/10 nM IIa.

The mixture was then incubated at 37° C. and then at various times 0, 0.5, 1, 2, 3.5, 6 and 8 h, and a 20 μl aliquot was removed and deposited in a well of a flat-bottomed microplate containing 180 μl of r-hirudin at 50 nM. The pNAPEP 1065 substrate (50 μl) diluted to ½ in WFI water was added and an immediate reading in kinetic mode was taken every 25 seconds for 10 min at 405 nm.

2. Results

The incubation of the plasma factor X in the presence of thrombin did not result in the appearance of FXa activity, confirming that this molecule cannot be activated by thrombin.

On the other hand, the FVIIv1-psFX-IIa-F2-Fc molecule produced in three cell lines HEK293, CHO-S and YB2/0 were sensitive to this activation and caused FXa to appear in a linear manner over time. The amount of FXa generated was slightly greater with the product from YB2/0.

These data indicate that the presence of a propeptide different than that of factor X does not impair the capacity of the molecule to be activated by thrombin.

### EXAMPLE 13

### Measurement in Terms of Thrombin Generation Time (TGT) of the Procoagulant Capacity of the FX-IIa-F2-Fcs: Activation of the Extrinsic Coagulation Pathway (TF 1 pM/PL 4 μM) in FVIII-Deficient Plasma

1. Protocol

A protocol identical to that described in example 8 was applied.

2. Results

The controls, factor VIII-deficient plasma reconstituted with 0.1 U/ml of factor VIII (□) or 1 U/ml of factor VIII (▪) allow thrombin generation which increases as a function of the amount of FVIII. A normal plasma gives a median signal between these two conditions (●). The presence of the FVIIv1-psFX-IIa-F2-Fc molecule (4 μg/ml) makes it possible to correct the FVIII deficiency. The factor produced in HEK293 gives a more powerful signal than that of the normal plasma with an identical lag time. However, it is slightly greater than that of the deficient plasma+1 U/ml of factor VIII.

The product from YB2/0 itself also gives a powerful signal, but with a further increased lag time. The product from CHO gives a more moderate signal with a further increased lag time, but capable of correcting the FVIII deficiency.

These data show that FVIIv1-psFX-IIa-F2-Fc having the propeptide as described in the sequence SEQ ID No.: 14 and produced in various cell lines has the capacity to restore a factor VIII deficiency.

1. A protein which is a factor X variant comprising a mutated sequence of SEQ ID No.: 1, wherein said mutated sequence of SEQ ID No.: 1 comprises a mutation A, A′, B, C or C′, wherein:
the mutation A consists of the substitution of amino acids 43 to 52 of the sequence SEQ ID No.: 1 by a sequence chosen from DFLAEGLTPR, KATN*ATLSPR and KATXATLSPR,
the mutation A′ consists of the substitution of amino acids 47 to 52 of the sequence SEQ ID No.: 1 by a sequence chosen from TSKLTR, FNDFTR, LSSMTR, PPSLTR and LSCGQR,
the mutation B consists of the insertion of a sequence chosen from DFLAEGLTPR, KATN*ATLSPR, KATXATLSPR, TSKLTR, FNDFTR, LSSMTR, PPSLTR and LSCGQR, between amino acids 52 and 53 of the sequence SEQ ID No.: 1,
the mutation C consists of the insertion of a sequence chosen from DFLAEGLTPR, KATN*ATLSPR and KATXATLSPR, between amino acids 52 and 53 of the sequence SEQ ID No.: 1, and of the deletion of amino acids 4 to 13 of the sequence SEQ ID No.: 1,
the mutation C′ consists of the insertion of a sequence chosen from TSKLTR, FNDFTR, LSSMTR, PPSLTR and LSCGQR, between amino acids 52 and 53 of the sequence SEQ ID No.: 1, and of the deletion of amino acids 4 to 9 of the sequence SEQ ID No.: 1,
wherein N* is an optionally glycosylated asparagine, and said protein comprising, at its N-terminal end, the signal peptide of sequence SEQ ID No.: 7 fused to a propeptide different than the natural propeptide of factor X.

2. The protein as claimed in claim 1, wherein the mutated sequence of SEQ ID No.: 1 comprises the mutation B.

3. The protein as claimed in claim 1, wherein the propeptide different than the natural propeptide of factor X is chosen from the thrombin propeptide, the factor VII propeptide, and the protein C propeptide, and the modified versions thereof.

4. The protein as claimed in claim 1, wherein the propeptide is chosen from the sequences SEQ ID No.: 13, SEQ ID No.: 14, SEQ ID No.: 15 and SEQ ID No.: 16.

5. The protein as claimed in claim 1, wherein the signal peptide of sequence SEQ ID No.: 7 fused to a propeptide different than the natural propeptide of factor X is chosen from the sequences SEQ ID No.: 18, SEQ ID No.: 19, SEQ ID No.: 20 and SEQ ID No.: 21.

6. The protein as claimed in claim 1, further comprising an intermediate sequence, between the signal peptide of sequence SEQ ID No.: 7 fused to a propeptide different than the natural propeptide of factor X, and the mutated sequence of SEQ ID No.: 1.

7. The protein as claimed in claim 6, wherein the intermediate sequence is the sequence of the factor X light chain, preferably in the sequence SEQ ID No.: 5.

8. The protein as claimed in claim 1, further comprising, from the N-terminal to C-terminal end:
the signal peptide of sequence SEQ ID No.: 7 fused to a propeptide different than the natural propeptide of factor X, then
the sequence SEQ ID No.: 5, then
said mutated sequence of SEQ ID No.: 1.

9. The protein as claimed in claim 1, further comprising from the N-terminal to C-terminal end:
the signal peptide of sequence SEQ ID No.: 7 fused to a propeptide different than the natural propeptide of factor X, then
the sequence SEQ ID No.: 5, then
the sequence SEQ ID No.: 11.

10. The protein as claimed in claim 1, further comprising a sequence chosen from SEQ ID No.: 22, SEQ ID No.: 23, SEQ ID No.: 24 and SEQ ID No.: 25.

11. The protein as claimed in claim 1, wherein the protein is fused, at the C-terminal end, to at least one wild-type Fc fragment or to at least one wild-type scFc fragment which is optionally mutated.

12. The protein as claimed in claim 11, wherein the wild-type Fc fragment has the sequence SEQ ID No.: 36 or SEQ ID No.: 37, optionally followed by a lysine in the C-terminal position.

13. The protein as claimed in claim 12, wherein the wild-type scFc fragment has the sequence SEQ ID No.: 42.

14. The protein as claimed in claim 11, wherein the protein has the sequence SEQ ID No.: 40 or SEQ ID No.: 43.

15. The protein as claimed in claim 11, wherein the wild-type Fc fragment or the wild-type scFc fragment is mutated so as to comprise the T366Y or Y407T mutation.

16. A nucleic acid that encodes the protein as claimed in claim 1.

17. The nucleic acid as claimed in claim 16, chosen from the sequences SEQ ID No.: 32, SEQ ID No.: 33, SEQ ID No.: 34 and SEQ ID No.: 35.

18. An expression cassette comprising the nucleic acid as claimed in claim 16.

19. An expression vector, comprising the expression cassette as claimed in claim 18.

20. The expression vector as claimed in claim 19, for use thereof as a medicament, preferably as a gene therapy medicament.

21. A recombinant cell comprising the nucleic acid as claimed in claim 16.

22. The protein as claimed in claim 1, for use thereof as a medicament.

23. The protein as claimed in claim 1, for use thereof for treating hemorrhagic disorders.

24. A method for producing a protein as claimed in claim 7, comprising:
a) the expression of a polycistronic, preferably bicistronic, vector, in a host cell, preferably a CHO cell, said vector comprising a polynucleotide encoding the protein, and a polynucleotide encoding the VKOR enzyme, preferably encoding the wild-type human vitamin K epoxide reductase subunit 1 complex (VKORC1), preferably in the presence of vitamin K;
b) the culturing of said host cell;
c) the recovery of the cell supernatant;
d) optionally at least one of the steps chosen from:
clarification of the supernatant, optionally followed by a filtration step,
concentration of the supernatant,
neutralization of the activated proteases by addition of protease inhibitors;
e) the purification of the protein as claimed in the invention by passing the production supernatant obtained in c) or d) over a column of aptamers capable of binding to the Gla domain of factor X.

25. A method for producing a protein as claimed in claim 7, comprising:
a) the expression of two expression vectors, one comprising a polynucleotide encoding the protein, and the other comprising a polynucleotide encoding the abovementioned VKOR enzyme, in a host cell, preferably a CHO cell, preferably in the presence of vitamin K;
b) the culturing of said host cell;
c) the recovery of the cell supernatant;
d) optionally at least one of the steps chosen from:
clarification of the supernatant, optionally followed by a filtration step,
concentration of the supernatant,
neutralization of the activated proteases by adding protease inhibitors;
e) the purification of the protein as claimed in the invention by passing the production supernatant obtained in c) or d) over a column of aptamers capable of binding to the Gla domain of factor X.


SEQUENCE LISTING (82 sequences)

SEQ ID NO: 1 - protein, 306 aa, Homo sapiens
SVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 2 - protein, 182 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKR

SEQ ID NO: 3 - protein, 52 aa, Homo sapiens
SVAQATSSSGEAPDSITWKPYDAADLDPTENPFDLLDFNQTQPERGDNNLTR

SEQ ID NO: 4 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 5 - protein, 142 aa, Homo sapiens
ANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKR

SEQ ID NO: 6 - protein, 254 aa, Homo sapiens [sequence omitted]

SEQ ID NO: 7 - protein, 31 aa, Homo sapiens
MGRPLHLVLLSASLAGLLLLGESLFIRREQA

SEQ ID NO: 8 - protein, 9 aa, Homo sapiens
NNILARVTR

SEQ ID NO: 9 - protein, 9 aa, Homo sapiens
NNILARVRR

SEQ ID NO: 10 - protein, 488 aa, Homo sapiens
MGRPLHLVLLSASLAGLLLLGESLFIRREQANNILARVRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQDFLAEGGGVRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 11 - protein, 316 aa, Artificial Sequence
  Mutant of Homo sapiens
SVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 12 - DNA, 40 bp, Artificial Sequence
  aptamer
ccacgacctcgcacatgacttgaagtaaaacgcgaattac

SEQ ID NO: 13 - protein, 19 aa, Homo sapiens
QHVFLAPQQARSLLQRVRR

SEQ ID NO: 14 - protein, 40 aa, Artificial Sequence
  FVIIv1 propeptide
AGGVAKASGGETRDMPWKPGPHRVFVTQEEAHGVLHRRRR

SEQ ID NO: 15 - protein, 18 aa, Artificial Sequence
  FVIIv2 propeptide
AVFVTQEEAHGVLHRRRR

SEQ ID NO: 16 - protein, 24 aa, Homo sapiens
TPAPLDSVFSSSERAHQVLRIRKR

SEQ ID NO: 17 - protein, 458 aa, Artificial Sequence
  FX-IIa
ANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 18 - protein, 50 aa, Artificial Sequence
  Human factor X signal peptide fused to the thrombin propeptide
MGRPLHLVLLSASLAGLLLLGESLFIRREQAQHVFLAPQQARSLLQRVRR

SEQ ID NO: 19 - protein, 71 aa, Artificial Sequence
  Human factor X signal peptide fused to FVIIv1
MGRPLHLVLLSASLAGLLLLGESLFIRREQAAGGVAKASGGETRDMPWKPGPHRVFVTQEEAHGVLHRRRR

SEQ ID NO: 20 - protein, 49 aa, Artificial Sequence
  Human vector X signal peptide fused to FVIIv2
MGRPLHLVLLSASLAGLLLLGESLFIRREQAAVFVTQEEAHGVLHRRRR

SEQ ID NO: 21 - protein, 55 aa, Artificial Sequence
  Human factor X signal peptide fused to the thrombin propeptide
MGRPLHLVLLSASLAGLLLLGESLFIRREQATPAPLDSVFSSSERAHQVLRIRKR

SEQ ID NO: 22 - protein, 508 aa, Artificial Sequence
  Human factor X signal peptide fused to the thrombin propeptide fused to FX-IIa
MGRPLHLVLLSASLAGLLLLGESLFIRREQAQHVFLAPQQARSLLQRVRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 23 - protein, 529 aa, Artificial Sequence
  Human factor X signal peptide fused to FVIIv1 fused to FX-IIa
MGRPLHLVLLSASLAGLLLLGESLFIRREQAAGGVAKASGGETRDMPWKPGPHRVFVTQEEAHGVLHRRRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 24 - protein, 507 aa, Artificial Sequence
  Human vector X signal peptide fused to FVIIv2 fused to FX-IIa
MGRPLHLVLLSASLAGLLLLGESLFIRREQAAVFVTQEEAHGVLHRRRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 25 - protein, 513 aa, Artificial Sequence
  Human factor X signal peptide fused to the thrombin propeptide fused to FX-IIa
MGRPLHLVLLSASLAGLLLLGESLFIRREQATPAPLDSVFSSSERAHQVLRIRKRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 26 - DNA, 93 bp, Artificial Sequence
  Nucleic sequence encoding Human factor X signal peptide
atgggaagacccctgcatctggtgctgctgtccgcctcactggctgggctgctgctgctgggagaatctctgtttatccgacgggagcaggca

SEQ ID NO: 27 - DNA, 57 bp, Artificial Sequence
  Nucleic sequence encoding Thrombin propeptide
cagcatgtcttcctggcaccacagcaggcacgaagtctgctgcagcgggtgcggaga

SEQ ID NO: 28 - DNA, 120 bp, Artificial Sequence
  Nucleic sequence encoding Factor VII propeptide version 1 (“FVIIv1”)
gctggcggcgtcgctaaggcttctggaggcgagacccgggacatgccctggaagcctgggccacatagagtgttcgtgacccaggaagaggctcatggagtgctgcatagaaggagaagg

SEQ ID NO: 29 - DNA, 54 bp, Artificial Sequence
  Nucleic sequence encoding Factor VII propeptide version 2 (“FVIIv2”)
gctgtgttcgtgacccaggaagaggctcatggagtgctgcatagaaggagaagg

SEQ ID NO: 30 - DNA, 72 bp, Artificial Sequence
  Nucleic sequence encoding Protein C propeptide
acacccgcccctctggatagcgtgttcagcagctctgagcgggcccaccaggtgctgcggatcagaaagaga

SEQ ID NO: 31 - DNA, 1374 bp, Artificial Sequence
  Nucleic sequence encoding FX-IIa
gccaactctttcctggaggaaatgaagaaaggccacctggagcgggaatgcatggaggaaacctgtagttacgaggaagccagagaggtgttcgaagactcagataagacaaacgagttttggaataagtacaaagacggcgatcagtgcgaaactagcccatgtcagaaccaggggaagtgcaaagatggactgggcgagtacacctgcacatgtctggagggattcgaaggcaagaattgcgaactgtttaccagaaagctgtgctccctggataacggcgactgcgatcagttttgtcatgaggaacagaattccgtggtctgctcttgtgccaggggatacacactggctgacaatggcaaggcatgcatccccaccggcccctatccttgtgggaagcagacactggagaggcgcaaaaggtcagtggctcaggcaactagctcctctggcgaggcccccgatagcattacctggaaaccttatgacgccgctgacctggaccccacagagaacccctttgacctgctggacttcaaccagacacagcctgaaagaggcgataacaatctgactagggacttcctggccgagggcctgacccctaggatcgtgggaggacaggagtgcaaggacggagaatgtccatggcaggccctgctgattaacgaggaaaatgagggattctgcggaggcactatcctgagcgagttctacattctgaccgcagcccactgtctgtatcaggctaagcgattcaaagtgcgggtcggcgacagaaacaccgagcaggaggaagggggagaagcagtgcacgaggtcgaagtggtcatcaagcataatcgcttcactaaagagacctacgactttgatatcgctgtgctgcgcctgaagacacctattactttccgaatgaacgtcgcccctgcttgcctgccagagcgagattgggccgaaagcaccctgatgacacagaaaactggcatcgtgagcgggtttggacggacacatgagaagggcaggcagtccactcgcctgaaaatgctggaagtgccctacgtcgaccggaactcttgtaagctgagtagcagcttcatcattacccagaatatgttttgcgccgggtatgacacaaagcaggaggatgcttgtcagggagacagtggcgggcctcacgtgactaggttcaaagatacttattttgtgaccggcatcgtcagctggggagagggatgcgcacgcaaggggaaatacggaatctataccaaggtgacagcctttctgaaatggattgaccgatctatgaagacccgggggctgccaaaggcaaaaagtcatgcccccgaggtcattaccagttcccctctgaaa

SEQ ID NO: 32 - DNA, 1524 bp, Artificial Sequence
  Nucleic sequence encoding Factor X variant
atgggaagacccctgcatctggtgctgctgtccgcctcactggctgggctgctgctgctgggagaatctctgtttatccgacgggagcaggcacagcatgtcttcctggcaccacagcaggcacgaagtctgctgcagcgggtgcggagagccaactctttcctggaggaaatgaagaaaggccacctggagcgggaatgcatggaggaaacctgtagttacgaggaagccagagaggtgttcgaagactcagataagacaaacgagttttggaataagtacaaagacggcgatcagtgcgaaactagcccatgtcagaaccaggggaagtgcaaagatggactgggcgagtacacctgcacatgtctggagggattcgaaggcaagaattgcgaactgtttaccagaaagctgtgctccctggataacggcgactgcgatcagttttgtcatgaggaacagaattccgtggtctgctcttgtgccaggggatacacactggctgacaatggcaaggcatgcatccccaccggcccctatccttgtgggaagcagacactggagaggcgcaaaaggtcagtggctcaggcaactagctcctctggcgaggcccccgatagcattacctggaaaccttatgacgccgctgacctggaccccacagagaacccctttgacctgctggacttcaaccagacacagcctgaaagaggcgataacaatctgactagggacttcctggccgagggcctgacccctaggatcgtgggaggacaggagtgcaaggacggagaatgtccatggcaggccctgctgattaacgaggaaaatgagggattctgcggaggcactatcctgagcgagttctacattctgaccgcagcccactgtctgtatcaggctaagcgattcaaagtgcgggtcggcgacagaaacaccgagcaggaggaagggggagaagcagtgcacgaggtcgaagtggtcatcaagcataatcgcttcactaaagagacctacgactttgatatcgctgtgctgcgcctgaagacacctattactttccgaatgaacgtcgcccctgcttgcctgccagagcgagattgggccgaaagcaccctgatgacacagaaaactggcatcgtgagcgggtttggacggacacatgagaagggcaggcagtccactcgcctgaaaatgctggaagtgccctacgtcgaccggaactcttgtaagctgagtagcagcttcatcattacccagaatatgttttgcgccgggtatgacacaaagcaggaggatgcttgtcagggagacagtggcgggcctcacgtgactaggttcaaagatacttattttgtgaccggcatcgtcagctggggagagggatgcgcacgcaaggggaaatacggaatctataccaaggtgacagcctttctgaaatggattgaccgatctatgaagacccgggggctgccaaaggcaaaaagtcatgcccccgaggtcattaccagttcccctctgaaa

SEQ ID NO: 33 - DNA, 1587 bp, Artificial Sequence
  Nucleic sequence encoding Factor X variant
atgggaagacccctgcatctggtgctgctgtccgcctcactggctgggctgctgctgctgggagaatctctgtttatccgacgggagcaggcagctggcggcgtcgctaaggcttctggaggcgagacccgggacatgccctggaagcctgggccacatagagtgttcgtgacccaggaagaggctcatggagtgctgcatagaaggagaagggccaactctttcctggaggaaatgaagaaaggccacctggagcgggaatgcatggaggaaacctgtagttacgaggaagccagagaggtgttcgaagactcagataagacaaacgagttttggaataagtacaaagacggcgatcagtgcgaaactagcccatgtcagaaccaggggaagtgcaaagatggactgggcgagtacacctgcacatgtctggagggattcgaaggcaagaattgcgaactgtttaccagaaagctgtgctccctggataacggcgactgcgatcagttttgtcatgaggaacagaattccgtggtctgctcttgtgccaggggatacacactggctgacaatggcaaggcatgcatccccaccggcccctatccttgtgggaagcagacactggagaggcgcaaaaggtcagtggctcaggcaactagctcctctggcgaggcccccgatagcattacctggaaaccttatgacgccgctgacctggaccccacagagaacccctttgacctgctggacttcaaccagacacagcctgaaagaggcgataacaatctgactagggacttcctggccgagggcctgacccctaggatcgtgggaggacaggagtgcaaggacggagaatgtccatggcaggccctgctgattaacgaggaaaatgagggattctgcggaggcactatcctgagcgagttctacattctgaccgcagcccactgtctgtatcaggctaagcgattcaaagtgcgggtcggcgacagaaacaccgagcaggaggaagggggagaagcagtgcacgaggtcgaagtggtcatcaagcataatcgcttcactaaagagacctacgactttgatatcgctgtgctgcgcctgaagacacctattactttccgaatgaacgtcgcccctgcttgcctgccagagcgagattgggccgaaagcaccctgatgacacagaaaactggcatcgtgagcgggtttggacggacacatgagaagggcaggcagtccactcgcctgaaaatgctggaagtgccctacgtcgaccggaactcttgtaagctgagtagcagcttcatcattacccagaatatgttttgcgccgggtatgacacaaagcaggaggatgcttgtcagggagacagtggcgggcctcacgtgactaggttcaaagatacttattttgtgaccggcatcgtcagctggggagagggatgcgcacgcaaggggaaatacggaatctataccaaggtgacagcctttctgaaatggattgaccgatctatgaagacccgggggctgccaaaggcaaaaagtcatgcccccgaggtcattaccagttcccctctgaaa

SEQ ID NO: 34 - DNA, 1521 bp, Artificial Sequence
  Nucleic sequence encoding Factor X variant
atgggaagacccctgcatctggtgctgctgtccgcctcactggctgggctgctgctgctgggagaatctctgtttatccgacgggagcaggcagctgtgttcgtgacccaggaagaggctcatggagtgctgcatagaaggagaagggccaactctttcctggaggaaatgaagaaaggccacctggagcgggaatgcatggaggaaacctgtagttacgaggaagccagagaggtgttcgaagactcagataagacaaacgagttttggaataagtacaaagacggcgatcagtgcgaaactagcccatgtcagaaccaggggaagtgcaaagatggactgggcgagtacacctgcacatgtctggagggattcgaaggcaagaattgcgaactgtttaccagaaagctgtgctccctggataacggcgactgcgatcagttttgtcatgaggaacagaattccgtggtctgctcttgtgccaggggatacacactggctgacaatggcaaggcatgcatccccaccggcccctatccttgtgggaagcagacactggagaggcgcaaaaggtcagtggctcaggcaactagctcctctggcgaggcccccgatagcattacctggaaaccttatgacgccgctgacctggaccccacagagaacccctttgacctgctggacttcaaccagacacagcctgaaagaggcgataacaatctgactagggacttcctggccgagggcctgacccctaggatcgtgggaggacaggagtgcaaggacggagaatgtccatggcaggccctgctgattaacgaggaaaatgagggattctgcggaggcactatcctgagcgagttctacattctgaccgcagcccactgtctgtatcaggctaagcgattcaaagtgcgggtcggcgacagaaacaccgagcaggaggaagggggagaagcagtgcacgaggtcgaagtggtcatcaagcataatcgcttcactaaagagacctacgactttgatatcgctgtgctgcgcctgaagacacctattactttccgaatgaacgtcgcccctgcttgcctgccagagcgagattgggccgaaagcaccctgatgacacagaaaactggcatcgtgagcgggtttggacggacacatgagaagggcaggcagtccactcgcctgaaaatgctggaagtgccctacgtcgaccggaactcttgtaagctgagtagcagcttcatcattacccagaatatgttttgcgccgggtatgacacaaagcaggaggatgcttgtcagggagacagtggcgggcctcacgtgactaggttcaaagatacttattttgtgaccggcatcgtcagctggggagagggatgcgcacgcaaggggaaatacggaatctataccaaggtgacagcctttctgaaatggattgaccgatctatgaagacccgggggctgccaaaggcaaaaagtcatgcccccgaggtcattaccagttcccctctgaaa

SEQ ID NO: 35 - DNA, 1539 bp, Artificial Sequence
  Nucleic sequence encoding Factor X variant
atgggaagacccctgcatctggtgctgctgtccgcctcactggctgggctgctgctgctgggagaatctctgtttatccgacgggagcaggcaacacccgcccctctggatagcgtgttcagcagctctgagcgggcccaccaggtgctgcggatcagaaagagagccaactctttcctggaggaaatgaagaaaggccacctggagcgggaatgcatggaggaaacctgtagttacgaggaagccagagaggtgttcgaagactcagataagacaaacgagttttggaataagtacaaagacggcgatcagtgcgaaactagcccatgtcagaaccaggggaagtgcaaagatggactgggcgagtacacctgcacatgtctggagggattcgaaggcaagaattgcgaactgtttaccagaaagctgtgctccctggataacggcgactgcgatcagttttgtcatgaggaacagaattccgtggtctgctcttgtgccaggggatacacactggctgacaatggcaaggcatgcatccccaccggcccctatccttgtgggaagcagacactggagaggcgcaaaaggtcagtggctcaggcaactagctcctctggcgaggcccccgatagcattacctggaaaccttatgacgccgctgacctggaccccacagagaacccctttgacctgctggacttcaaccagacacagcctgaaagaggcgataacaatctgactagggacttcctggccgagggcctgacccctaggatcgtgggaggacaggagtgcaaggacggagaatgtccatggcaggccctgctgattaacgaggaaaatgagggattctgcggaggcactatcctgagcgagttctacattctgaccgcagcccactgtctgtatcaggctaagcgattcaaagtgcgggtcggcgacagaaacaccgagcaggaggaagggggagaagcagtgcacgaggtcgaagtggtcatcaagcataatcgcttcactaaagagacctacgactttgatatcgctgtgctgcgcctgaagacacctattactttccgaatgaacgtcgcccctgcttgcctgccagagcgagattgggccgaaagcaccctgatgacacagaaaactggcatcgtgagcgggtttggacggacacatgagaagggcaggcagtccactcgcctgaaaatgctggaagtgccctacgtcgaccggaactcttgtaagctgagtagcagcttcatcattacccagaatatgttttgcgccgggtatgacacaaagcaggaggatgcttgtcagggagacagtggcgggcctcacgtgactaggttcaaagatacttattttgtgaccggcatcgtcagctggggagagggatgcgcacgcaaggggaaatacggaatctataccaaggtgacagcctttctgaaatggattgaccgatctatgaagacccgggggctgccaaaggcaaaaagtcatgcccccgaggtcattaccagttcccctctgaaa

SEQ ID NO: 36 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 231 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 10 aa, Homo sapiens
DKTHTCPPCP

SEQ ID NO: 39 - protein, 15 aa, Homo sapiens
EPKSSDKTHTCPPCP

SEQ ID NO: 40 - protein, 755 aa, Artificial Sequence
  Human factor X signal peptide fused to FVIIv1 fused to FX-IIa fused to the wild-type Fc fragment
MGRPLHLVLLSASLAGLLLLGESLFIRREQAAGGVAKASGGETRDMPWKPGPHRVFVTQEEAHGVLHRRRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - DNA, 2265 bp, Artificial Sequence
  Nucleic acid encoding Human factor X signal peptide fused to FVIIv1 fused to FX-IIa fused to the wild -type Fc fragment
atgggaagacccctgcatctggtgctgctgtccgcctcactggctgggctgctgctgctgggagaatctctgtttatccgacgggagcaggcagctggcggcgtcgctaaggcttctggaggcgagacccgggacatgccctggaagcctgggccacatagagtgttcgtgacccaggaagaggctcatggagtgctgcatagaaggagaagggccaactctttcctggaggaaatgaagaaaggccacctggagcgggaatgcatggaggaaacctgtagttacgaggaagccagagaggtgttcgaagactcagataagacaaacgagttttggaataagtacaaagacggcgatcagtgcgaaactagcccatgtcagaaccaggggaagtgcaaagatggactgggcgagtacacctgcacatgtctggagggattcgaaggcaagaattgcgaactgtttaccagaaagctgtgctccctggataacggcgactgcgatcagttttgtcatgaggaacagaattccgtggtctgctcttgtgccaggggatacacactggctgacaatggcaaggcatgcatccccaccggcccctatccttgtgggaagcagacactggagaggcgcaaaaggtcagtggctcaggcaactagctcctctggcgaggcccccgatagcattacctggaaaccttatgacgccgctgacctggaccccacagagaacccctttgacctgctggacttcaaccagacacagcctgaaagaggcgataacaatctgactagggacttcctggccgagggcctgacccctaggatcgtgggaggacaggagtgcaaggacggagaatgtccatggcaggccctgctgattaacgaggaaaatgagggattctgcggaggcactatcctgagcgagttctacattctgaccgcagcccactgtctgtatcaggctaagcgattcaaagtgcgggtcggcgacagaaacaccgagcaggaggaagggggagaagcagtgcacgaggtcgaagtggtcatcaagcataatcgcttcactaaagagacctacgactttgatatcgctgtgctgcgcctgaagacacctattactttccgaatgaacgtcgcccctgcttgcctgccagagcgagattgggccgaaagcaccctgatgacacagaaaactggcatcgtgagcgggtttggacggacacatgagaagggcaggcagtccactcgcctgaaaatgctggaagtgccctacgtcgaccggaactcttgtaagctgagtagcagcttcatcattacccagaatatgttttgcgccgggtatgacacaaagcaggaggatgcttgtcagggagacagtggcgggcctcacgtgactaggttcaaagatacttattttgtgaccggcatcgtcagctggggagagggatgcgcacgcaaggggaaatacggaatctataccaaggtgacagcctttctgaaatggattgaccgatctatgaagacccgggggctgccaaaggcaaaaagtcatgcccccgaggtcattaccagttcccctctgaaagacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccagga

SEQ ID NO: 42 - protein, 462 aa, Artificial Sequence
  scFc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 991 aa, Artificial Sequence
  Human factor X signal peptide fused to FVIIv1 fused to FX-IIa fused to the wild-type scFc fragment
MGRPLHLVLLSASLAGLLLLGESLFIRREQAAGGVAKASGGETRDMPWKPGPHRVFVTQEEAHGVLHRRRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRDFLAEGLTPRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - DNA, 2973 bp, Artificial Sequence
  Nucleic acid encoding Human factor X signal peptide fused to FVIIv1 fused to FX-IIa fused to the wild -type scFc fragment
atgggaagacccctgcatctggtgctgctgtccgcctcactggctgggctgctgctgctgggagaatctctgtttatccgacgggagcaggcagctggcggcgtcgctaaggcttctggaggcgagacccgggacatgccctggaagcctgggccacatagagtgttcgtgacccaggaagaggctcatggagtgctgcatagaaggagaagggccaactctttcctggaggaaatgaagaaaggccacctggagcgggaatgcatggaggaaacctgtagttacgaggaagccagagaggtgttcgaagactcagataagacaaacgagttttggaataagtacaaagacggcgatcagtgcgaaactagcccatgtcagaaccaggggaagtgcaaagatggactgggcgagtacacctgcacatgtctggagggattcgaaggcaagaattgcgaactgtttaccagaaagctgtgctccctggataacggcgactgcgatcagttttgtcatgaggaacagaattccgtggtctgctcttgtgccaggggatacacactggctgacaatggcaaggcatgcatccccaccggcccctatccttgtgggaagcagacactggagaggcgcaaaaggtcagtggctcaggcaactagctcctctggcgaggcccccgatagcattacctggaaaccttatgacgccgctgacctggaccccacagagaacccctttgacctgctggacttcaaccagacacagcctgaaagaggcgataacaatctgactagggacttcctggccgagggcctgacccctaggatcgtgggaggacaggagtgcaaggacggagaatgtccatggcaggccctgctgattaacgaggaaaatgagggattctgcggaggcactatcctgagcgagttctacattctgaccgcagcccactgtctgtatcaggctaagcgattcaaagtgcgggtcggcgacagaaacaccgagcaggaggaagggggagaagcagtgcacgaggtcgaagtggtcatcaagcataatcgcttcactaaagagacctacgactttgatatcgctgtgctgcgcctgaagacacctattactttccgaatgaacgtcgcccctgcttgcctgccagagcgagattgggccgaaagcaccctgatgacacagaaaactggcatcgtgagcgggtttggacggacacatgagaagggcaggcagtccactcgcctgaaaatgctggaagtgccctacgtcgaccggaactcttgtaagctgagtagcagcttcatcattacccagaatatgttttgcgccgggtatgacacaaagcaggaggatgcttgtcagggagacagtggcgggcctcacgtgactaggttcaaagatacttattttgtgaccggcatcgtcagctggggagagggatgcgcacgcaaggggaaatacggaatctataccaaggtgacagcctttctgaaatggattgaccgatctatgaagacccgggggctgccaaaggcaaaaagtcatgcccccgaggtcattaccagttcccctctgaaagacaaaacccatacatgcccaccttgtccagcacctgaactgctgggaggaccatccgtgttcctgtttccacccaagcccaaagatacactgatgattagtcggacccctgaggtgacatgcgtggtcgtggatgtctcacacgaggacccagaagtgaagtttaactggtacgtggacggcgtggaagtccataatgccaagaccaaacctcgcgaggaacagtacaacagtacatatcgagtcgtgtcagtgctgactgtcctgcaccaggattggctgaacggaaaggagtataagtgcaaagtgagcaataaggctctgccagcacccatcgagaaaacaatttccaaggcaaaaggccagccaagggaaccccaggtgtacactctgcctccaagccgcgatgagctgacaaagaaccaggtgtccctgacttgtctggtcaaagggttctatccctccgacatcgccgtggagtgggaatctaatggacagcctgagaacaattacaagaccacaccccctgtgctggactcagatgggagcttctttctgtattctaagctgactgtggacaaaagtagatggcagcagggaaacgtgttttcttgcagtgtcatgcacgaggccctgcacaatcattacacccagaagtcactgagcctgtccccaggaggaggaggaggaagcgagcccaagagctccgataaaactcatacctgcccaccctgtcctgctccagaactgctgggaggccctagcgtgttcctgtttcctccaaagccaaaagacacactgatgatttctaggactcccgaggtgacctgcgtggtggtcgatgtcagtcacgaggaccctgaagtgaagttcaactggtacgtggatggagtcgaggtgcacaacgccaagaccaaaccccgggaggaacagtacaacagcacctatagagtggtctccgtgctgacagtcctgcaccaggactggctgaacgggaaggaatacaagtgcaaagtgtccaataaggccctgcccgctcctatcgaaaaaaccatttctaaggctaaaggccagccccgggagccacaggtgtacacactgcccccttctcgggatgaactgaccaagaaccaggtgagtctgacatgtctggtcaaaggcttctatccaagtgacatcgcagtggagtgggaatcaaatgggcagcccgagaacaattacaagactaccccacccgtgctggactccgatggctctttctttctgtattcaaagctgaccgtggacaaaagcagatggcagcaggggaacgtgttcagctgcagtgtcatgcacgaagcactgcacaatcattacactcagaaatcactgtcactgtcacctgga

SEQ ID NO: 45 - protein, 162 aa, Homo sapiens
MGSTWGSPGWVRLALCLTGLVLSLYALHVKAARARDRDYRALCDVGTAISCSRVFSSRWGRGFGLVEHVLGQDSILNQSNSIFGCIFYTLQLLLGCLRTRWASVLMLLSSLVSLAGSVYLAWILFFVLYDFCIVCITTYAINVSLMWLSFRKVQEPQGKAKR

SEQ ID NO: 46 - DNA, 486 bp, Homo sapiens
atgggatcaacatgggggtcacctggctgggtccgactggccctgtgcctgactggactggtcctgtctctgtatgccctgcacgtcaaggccgctcgggctagagacagggattatcgcgcactgtgcgacgtgggaacagccatctcatgtagcagggtcttcagctcccggtggggcagagggtttggactggtggagcacgtcctgggccaggatagcattctgaaccagtccaattctatcttcggctgcatcttctacactctgcagctgctgctgggatgtctgcgaacccgatgggcttccgtgctgatgctgctgtctagtctggtgagtctggcagggtcagtctacctggcctggattctgttcttcgtgctgtatgacttctgcatcgtctgtattaccacatacgccattaacgtgtctctgatgtggctgtcattcagaaaggtccaggaaccccagggaaaggcaaaaagg

SEQ ID NO: 47 - protein, 24 aa, Homo sapiens
MAHVRGLQLPGCLALAALCSLVHS

SEQ ID NO: 48 - protein, 20 aa, Homo sapiens
MVSQALRLLCLLLGLQGCLA

SEQ ID NO: 49 - protein, 18 aa, Homo sapiens
MWQLTSLLLFVATWGISG

SEQ ID NO: 50 - DNA, 23 bp, Artificial Sequence
  primer
accagctgctagcaagcttgccg

SEQ ID NO: 51 - DNA, 54 bp, Artificial Sequence
  primer
gtcaggccctcggccaggaagtccctagtcagattgttatcgcctctttcaggc

SEQ ID NO: 52 - DNA, 43 bp, Artificial Sequence
  primer
agggcctgacccctaggatcgtgggaggacaggagtgcaagga

SEQ ID NO: 53 - DNA, 39 bp, Artificial Sequence
  primer
gaaactatttaaatggatcctcacttgccgtcaatcagc

SEQ ID NO: 54 - DNA, 21 bp, Artificial Sequence
  primer
ggtggcggcaagcttgctagc

SEQ ID NO: 55 - DNA, 29 bp, Artificial Sequence
  primer
ccttgggcaataaatactagtggcgttac

SEQ ID NO: 56 - DNA, 36 bp, Artificial Sequence
  primer
aagcttgccgccaccatggctcacgtccgagggctg

SEQ ID NO: 57 - DNA, 46 bp, Artificial Sequence
  primer
cttcatttcctccaggaaagagttggctctccgcacccgctgcagc

SEQ ID NO: 58 - DNA, 37 bp, Artificial Sequence
  primer
aagcttgccgccaccatggtgtctcaggctctgcggc

SEQ ID NO: 59 - DNA, 42 bp, Artificial Sequence
  primer
caggaaagagttggcccttctccttctatgcagcactccatg

SEQ ID NO: 60 - DNA, 37 bp, Artificial Sequence; identical to SEQ ID NO: 58
  primer

SEQ ID NO: 61 - DNA, 34 bp, Artificial Sequence
  primer
gtcacgaacacagcagccagacatccctgcagtc

SEQ ID NO: 62 - DNA, 56 bp, Artificial Sequence
  primer
aagcttgccgccaccatgtggcagctgaccagcctgctgctgttcgtggccacatg

SEQ ID NO: 63 - DNA, 60 bp, Artificial Sequence
  primer
gagctgctgaacacgctatccagaggggcgggtgtgccagagatgccccatgtggccacg

SEQ ID NO: 64 - DNA, 60 bp, Artificial Sequence
  primer
ttcagcagctctgagcgggcccaccaggtgctgcggatcagaaagagagccaactctttc

SEQ ID NO: 65 - DNA, 27 bp, Artificial Sequence
  primer
gccaactctttcctggaggaaatgaag

SEQ ID NO: 66 - DNA, 33 bp, Artificial Sequence
  primer
agctctagacaattgatttaaatggatcctcac

SEQ ID NO: 67 - DNA, 22 bp, Artificial Sequence
  primer
gtggagactgaagttaggccag

SEQ ID NO: 68 - DNA, 22 bp, Artificial Sequence
  primer
gtggagactgaagttaggccag

SEQ ID NO: 69 - DNA, 21 bp, Artificial Sequence
  primer
ggaggcactatcctgagcgag

SEQ ID NO: 70 - DNA, 33 bp, Artificial Sequence; identical to SEQ ID NO: 66
  primer

SEQ ID NO: 71 - DNA, 38 bp, Artificial Sequence
  primer
gacgggagcaggcccagcatgtcttcctggcaccacag

SEQ ID NO: 72 - DNA, 33 bp, Artificial Sequence; identical to SEQ ID NO: 66
  primer

SEQ ID NO: 73 - DNA, 32 bp, Artificial Sequence
  primer
cgggagcaggccgctggcggcgtcgctaaggc

SEQ ID NO: 74 - DNA, 33 bp, Artificial Sequence; identical to SEQ ID NO: 66
  primer

SEQ ID NO: 75 - DNA, 36 bp, Artificial Sequence
  primer
cgggagcaggccgctgtgttcgtgacccaggaagag

SEQ ID NO: 76 - DNA, 33 bp, Artificial Sequence; identical to SEQ ID NO: 66
  primer

SEQ ID NO: 77 - DNA, 34 bp, Artificial Sequence
  primer
cgggagcaggccacacccgcccctctggatagcg

SEQ ID NO: 78 - DNA, 22 bp, Artificial Sequence
  primer
gtggagactgaagttaggccag

SEQ ID NO: 79 - DNA, 22 bp, Artificial Sequence
  primer
gtggagactgaagttaggccag

SEQ ID NO: 80 - DNA, 21 bp, Artificial Sequence
  primer
ggaggcactatcctgagcgag

SEQ ID NO: 81 - protein, 15 aa, Homo sapiens
EPKSCDKTHTCPPCP

SEQ ID NO: 82 - DNA, 2268 bp, Artificial Sequence
  Optimized nucleic acid sequence encoding Human factor X signal peptide fused to FVIIv1 fused to FX-II a fused to the wild-type scFc fragment
atgggcagacctctgcacctggtgctgctgtctgcctctctggctggactgctgctgctgggcgagagcctgttcatcagaagagagcaggccgctggcggagtggccaaagcttctggcggcgagacaagagacatgccctggaagcctggcccccacagagtgttcgtgacccaggaagaggcccacggcgtgctgcacagaagaagaagggccaacagcttcctggaagagatgaagaagggacacctggaacgcgagtgcatggaagaaacctgcagctacgaagaggccagagaagtgttcgaggacagcgacaagaccaacgagttctggaacaagtacaaggacggcgaccagtgcgaaaccagcccctgccagaatcagggcaagtgcaaggatggcctgggcgagtacacctgtacctgcctggaaggctttgagggcaagaactgcgagctgttcacccggaagctgtgcagcctggacaacggcgactgcgaccagttctgccacgaggaacagaacagcgtcgtgtgcagctgcgccagaggctacaccctggccgataatggcaaggcctgcatccccaccggcccttacccttgtggcaagcagaccctggaacggcggaagagatctgtggcccaggctacaagcagcagcggcgaggcccctgatagcatcacatggaagccctacgacgccgccgacctggaccctaccgagaaccctttcgacctgctggacttcaaccagacccagcccgagcggggcgacaacaacctgaccagagatttcctggccgagggcctgacccccagaatcgtgggaggacaggaatgcaaggacggggagtgtccttggcaggccctgctgatcaacgaggaaaacgagggcttctgcggcggcaccatcctgagcgagttctacatcctgacagccgcccactgcctgtaccaggccaagcggttcaaagtgcgcgtgggcgacagaaacaccgagcaggaagagggcggagaggccgtgcacgaagtggaagtcgtgatcaagcacaaccggttcaccaaagagacatacgacttcgacattgccgtgctgcggctgaaaacccccatcaccttccggatgaacgtggcccctgcctgtctgcccgaaagagattgggccgagagcaccctgatgacccagaaaaccggcatcgtgtccggcttcggccggacacacgaaaagggccggcagagcacccggctgaagatgctggaagtgccctacgtggaccggaacagctgcaagctgagcagcagcttcatcatcacccagaatatgttctgcgccggctacgacaccaaacaggaagatgcctgccagggcgactctggcggacctcacgtgaccagattcaaggacacctacttcgtgacagggatcgtgtcctggggcgagggctgtgccagaaaggggaagtacggcatctataccaaagtgaccgccttcctgaagtggatcgaccggtccatgaagaccaggggactgcccaaggccaagagccacgcccctgaagtgatcaccagcagccccctgaaggacaagacccacacctgtcccccttgccctgcccctgaactgctgggaggccctagcgtgttcctgttccccccaaagcccaaggatacactgatgatcagccggacccccgaagtgacctgcgtggtggtggatgtgtcccacgaggacccagaagtgaagttcaattggtacgtggacggcgtggaagtgcacaacgccaagaccaagcccagagaggaacagtacaactccacctaccgggtggtgtccgtgctgacagtgctgcaccaggactggctgaacggcaaagagtacaagtgcaaagtgtccaacaaggccctgcctgcccccatcgagaaaaccatctctaaggccaagggacagccccgcgagccccaggtgtacacactgcctccaagcagggacgagctgaccaagaatcaggtgtcactgacctgtctcgtgaagggcttctaccccagcgatattgccgtggaatgggagagcaacggccagcctgagaacaactacaagaccaccccccctgtgctggactccgacggctcattcttcctgtacagcaagctgaccgtggacaagagccggtggcagcagggcaacgtgttcagctgcagcgtgatgcacgaggccctgcacaaccactacacacagaagtccctgagcctgagccccggctga